(19) United States
(12) Patent Application Publication
(10) Pub. No.: US 2023/0272428 A1
(43) Pub. Date: Aug. 31, 2023

(54) METHODS AND COMPOSITIONS FOR CORRECTION OF DMD MUTATIONS

(71) Applicant: President and Fellows of Harvard College, Cambridge, MA (US)

(72) Inventor: Amy J. Wagers, Cambridge, MA (US)

(21) Appl. No.: 17/786,517

(22) PCT Filed: Dec. 16, 2020
(86) PCT No.: PCT/US2020/065437

§ 371 Date: Jun. 16, 2022

Related U.S. Application Data
(60) Provisional application No. 62/948,665, filed on Dec. 16, 2019.

(51) Int. Cl. C12N 15/90; C12N 9/22; C12N 15/11; C12N 15/86; A61K 38/46; A61K 31/7088; A61K 48/00; A61P 21/00

(52) U.S. Cl. CPC C12N 15/907; A61K 31/7088; A61K 38/465; A61K 48/0041; A61P 21/00; C12N 9/22; C12N 15/11; C12N 15/86; C12N 2310/20; C12N 2750/14143

(57) ABSTRACT

Disclosed herein are methods and compositions for treating muscular dystrophies, including DMD, by deleting and optionally replacing disease associated mutations in the genome of muscular dystrophy patients.

### RELATED APPLICATIONS

This application is the U.S. National Application of PCT/US2020/065437, filed Dec. 16, 2020, which claims the benefit of U.S. Provisional Application Serial No. 62/948,665, filed Dec. 16, 2019. The entire teachings of the above applications are incorporated herein by reference in their entirety. International Application No. PCT/US2020/065437 was published under PCT Article 21(2) in English.

### BACKGROUND OF THE INVENTION

Duchenne muscular dystrophy (DMD) occurs from a defect in the expression of the protein dystrophin. Approximately 60% of DMD patients have a mutation between exon 44 and 56 of the DMD gene. DMD results in significant movement disorders that may be seen in the first year of life. Most DMD patients rely on wheelchairs by 10-14 years and individuals with DMD typically die from breathing and/or heart failure in the late teens or early 20s.

### SUMMARY OF THE INVENTION

With the aim of delivering an in vivo therapeutic that could effectively correct a large fraction of existing human DMD mutations (including those contained within the DMD “hotspot” from exon 45-55), disclosed herein is a dual-gRNA CRISPR/Cas9-based system that enables homology-independent integration at the DMD locus of a synthetic intron-free DNA sequence encoding exons 45-55. Successful excision and replacement of exons 45-55 via this approach results in restoration of expression of full-length dystrophin protein, while excision alone results in truncated dystrophin (which is also therapeutic) by removing frame-shifting exons and “re-framing” the mutant sequence to express a partially functional protein. Thus, this strategy results in two distinct outcomes, both of which have therapeutic potential.

Some aspects of the present disclosure are directed to a method for modifying the genome of a mammalian muscle or muscle precursor cell, comprising contacting the cell with a Cas protein and a first and second guide ribonucleic acid (gRNA), wherein the first gRNA hybridizes to a first target site located in intron 44 of DMD and the second gRNA hybridizes to a second target site located in intron 55 of DMD, thereby modifying the genome of the mammalian muscle or muscle precursor cell located between intron 44 and intron 55 of DMD.

In some embodiments, the modification of the genome comprises a deletion of the nucleotide sequence between intron 44 and intron 55 of DMD. In some embodiments, the method further comprises contacting the cell with template DNA comprising the nucleotide sequences of exons 45 to 55 of wild-type DMD. In some embodiments, the template DNA comprises portions of the first and second target sites flanking the nucleotide sequences of exons 45 to 55 of wild-type DMD. In some embodiments, the modification of the genome comprises replacement of the nucleotide sequence between intron 44 and intron 55 of DMD with template DNA comprising exons 45 to 55 of wild-type DMD. In some embodiments, the replacement of the nucleotide sequence between intron 44 and intron 55 of DMD occurs via non-homologous end joining (NHEJ).

In some embodiments, the genome of the mammalian muscle or muscle precursor cell located between intron 44 and intron 55 of DMD comprises a mutation associated with a disease or condition. In some embodiments, the disease or condition is Duchenne muscular dystrophy.

In some embodiments, the cell is a human cell. In some embodiments, the cell is an induced pluripotent stem cell derived from a cell of a subject with a muscular dystrophy and having a mutation located between intron 44 and intron 55 of the DMD gene. In some embodiments, the subject has Duchenne muscular dystrophy.

In some embodiments, the Cas protein is Cas9. In some embodiments, the cell is contacted with one or more viruses transducing the Cas protein, the first gRNA, the second gRNA, or the template DNA. In some embodiments, the one or more viruses are AAV viruses. In some embodiments, the cell is contacted with a first virus transducing a nucleic acid encoding the Cas protein in the cell and a second virus transducing a nucleic acid encoding the first gRNA, the second gRNA, and template DNA in the cell. In some embodiments, at least the first virus or second virus is an AAV virus.

In some embodiments, the cell with a modified genome expresses truncated functional dystrophin lacking an amino acid sequence coded by exons 45 to 55 of wild-type DMD. In some embodiments, the cell with a modified genome expresses full length dystrophin comprising amino acid sequences coded by exons 45 to 55 of wild-type DMD. In some embodiments, the cell is contacted in vitro, ex vivo, or in vivo. In some embodiments, the cell is contacted with a virus inducing a nucleic acid of SEQ ID NO: 26 or 27, or a portion thereof inducing one or more of one or two gRNAs and a template.

Some aspects of the present disclosure are directed to a method of treating a muscular dystrophy in a subject in need thereof, comprising contacting a muscle or muscle precursor cell of the patient with a Cas protein and a first and second guide ribonucleic acid (gRNA), wherein the first gRNA hybridizes to a first target site located in intron 44 of DMD and the second gRNA hybridizes to a second target site located in intron 55 of DMD, thereby modifying the genome of the cell, wherein the subject’s genome comprises a mutation located between intron 44 and intron 55 of the DMD gene. In some embodiments, the modification of the genome comprises a deletion of the nucleotide sequence between intron 44 and intron 55 of DMD.

In some embodiments, the method further comprises contacting the cell with template DNA comprising the nucleotide sequences of exons 45 to 55 of wild-type DMD. In some embodiments, the template DNA comprises a portion of the first and second target sites flanking the nucleotide sequences of exons 45 to 55 of wild-type DMD. In some embodiments, the modification of the genome comprises replacement of the nucleotide sequence between intron 44 and intron 55 of DMD with template DNA comprising exons 45 to 55 of wild-type DMD. In some embodiments, the replacement of the nucleotide sequence between intron 44 and intron 55 of DMD occurs via non-homologous end joining (NHEJ). In some embodiments, the muscular dystrophy is Duchenne muscular dystrophy.

In some embodiments, the Cas protein is Cas9 (e.g., saCas9, SauriCas9, KKH Cas9, spCas9). In some embodiments, the subject is administered one or more viruses transducing the Cas protein, the first gRNA, the second gRNA, or the template DNA. In some embodiments, the one or more viruses are AAV viruses. In some embodiments, the subject is administered a first virus transducing a nucleic acid encoding the Cas protein in the cell and a second virus transducing a nucleic acid encoding the first gRNA, the second gRNA, and the template DNA in the cell. In some embodiments, at least the first virus or second virus is an AAV virus.

In some embodiments, the cell with a modified genome expresses truncated dystrophin lacking an amino acid sequence coded by exons 45 to 55 of wild-type DMD. In some embodiments, the cell with a modified genome expresses full length dystrophin comprising amino acid sequences coded by exons 45 to 55 of wild-type DMD. In some embodiments, the cell is contacted ex vivo or in vivo. In some embodiments, the cell is contacted with a virus inducing a nucleic acid of SEQ ID NO: 26 or 27, or a portion thereof inducing one or more of one or two gRNAs and a template.

Some aspects of the present disclosure are directed to a method for modifying the genome of a mammalian muscle or muscle precursor cell, comprising contacting the cell with a Cas protein and a first and second guide ribonucleic acid (gRNA), wherein the first gRNA hybridizes to a first target site located in a first intron of DMD and the second gRNA hybridizes to a second target site located in another intron of DMD, thereby modifying the genome of the mammalian muscle or muscle precursor cell located between the first and second target site. In some embodiments, DMD exon 23 is located between the first and second target sites.

In some embodiments, the method further comprises contacting the cell with template DNA comprising the nucleotide sequences of DMD exons located between the first and second target sites. In some embodiments, the template DNA comprises a portion of the first and second target sites flanking the nucleotide sequences of DMD exons located between the first and second target sites. In some embodiments, the modification of the genome comprises replacement of the nucleotide sequence located between the first and second target sites with template DNA comprising the nucleotide sequences of DMD exons located between the first and second target sites. In some embodiments, the replacement of the nucleotide sequence occurs via non-homologous end joining (NHEJ).

In some embodiments, the genome of the cell located between the first and second target sites comprises a mutation associated with a disease or condition. In some embodiments, the disease or condition is Duchenne muscular dystrophy. In some embodiments, the cell is an induced pluripotent stem cell derived from a cell of a subject with a muscular dystrophy and having a mutation located between the first and second target sites. In some embodiments, the cell is contacted with one or more viruses transducing the Cas protein, the first gRNA, the second gRNA, or the template DNA. In some embodiments, the cell is contacted with a first virus transducing a nucleic acid encoding the Cas protein in the cell and a second virus transducing a nucleic acid encoding the first gRNA, the second gRNA, and the template DNA in the cell. In some embodiments, the first virus or second virus is an AAV virus. In some embodiments, the cell with a modified genome is capable of expressing functional truncated dystrophin. In some embodiments, the cell with a modified genome is capable of expressing full length dystrophin. In some embodiments, the cell is contacted in vitro, ex vivo, or in vivo.

Some aspects of the present disclosure are related to a composition comprising a first virus transducing a nucleic acid encoding a Cas protein in a cell and a second virus transducing a nucleic acid encoding a first gRNA, a second gRNA, and a template DNA in a cell, wherein the first gRNA hybridizes to a first target site located in a first intron of DMD and the second gRNA hybridizes to a second target site located in another intron of DMD, and wherein the template DNA codes for one or more DMD exons located between the first and second targets sites. In some embodiments, the template DNA further comprises a portion of the first and second target sites flanking the one or more exons.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The patent or application file contains at least one drawing executed in color. Copies of this patent or patent application publication with color drawings will be provided by the Office upon request and payment of the necessary fee.

FIG. 1 shows a schematic for deletion of mutant exon 23 with two AAV. The first AAV providing a sequence for SaCas9 and the second providing two gRNA with target sites flanking exon 23. Result shown is deletion of mutant exon, resulting in the production of functional truncated dystrophin.

FIG. 2 shows a schematic for deletion of mutant exon 23 with two AAV. The first AAV providing a sequence for SaCas9 and the second providing two gRNA with target sites flanking exon 23 as well as wild-type exon 23 sequence flanked by the target sites for the two gRNA. Result shown is deletion of mutant exon, resulting in the production of functional truncated dystrophin.

FIG. 3 shows a schematic for deletion and replacement of mutant exon 23 with two AAV. The first AAV providing a sequence for SaCas9 and the second AAV providing two gRNA with target sites flanking exon 23 as well as wild-type exon 23 sequence flanked by the target sites for the two gRNA. Result shown is deletion of mutant exon resulting in either the production of functional truncated dystrophin or a full dystrophin protein with mutant exon 23 replaced by NHEJ with wild-type exon 23 sequence from the second AAV.

FIG. 4 shows a schematic for deletion and replacement of mutant exon 23 with two AAV. See the explanation in FIG. 3 above. Schematic shown in FIG. 4 further provides for if the wild-type exon 23 sequence from the second AAV is inserted by NHEJ into genome in the wrong direction, the gRNA target sites will be reconstituted and the insert excised, resulting in production of functional truncated dystrophin.

FIG. 5 shows use of the exon deletion and replacement strategy detailed in FIGS. 1-4 for exons 45-55, which is the location of mutations in 60% of Duchenne patients. This strategy can result in the production of functional truncated dystrophin or full length dystrophin comprising replacement exons 45-55.

FIG. 6 shows results for human DMD Exon deletion and replacement strategy targeting mutation ‘hotspot’ at exon 45-55 in HEK293 cells.

FIG. 7 is a schematic showing the predicted ligation sequence from NHEJ of replacement exons 45-55 in the correct direction at the junction of the insert sequence and intron 55. Sequence data shown herein confirms successful insertion. PCR with primer pairs 605 and 606 shown in FIG. 7 will only result in PCR amplification product upon successful insertion of replacement exons 45-55 in the correct direction.

FIG. 8 is a schematic showing the predicted ligation sequence from NHEJ of replacement exons 45-55 in the correct direction at the junction of the insert sequence and intron 44. Sequence data shown herein confirms successful insertion. PCR with primer pairs 603 and 606 shown in FIG. 8.

FIG. 9 is a schematic showing the predicted ligation sequence from NHEJ of intron 44 and 55. Sequence data shown herein confirms deletion of exons 45-55. PCR with primer pairs 603 and 606 shown in FIG. 8.

FIG. 10 shows PCR amplification of deletion products.

FIG. 11 shows PCR amplification of the junction between the template and intron 55.

FIG. 12 shows PCR amplification of the junction between intron 44 and intron 55.

FIG. 13 shows the top five variants detected by deep amplicon sequencing of the junction between intron 44 and the template.

FIG. 14 is a schematic showing the sequence of hDMD (NG_012232.1) from intron 44 to intron 55.

FIG. 15 is a schematic showing the sequence of HITI B with the template DNA comprising exons 45-55 as well as portions of intron 44 and intron 55 flanking the exons and comprising gRNA target sequences.

FIG. 16 is a schematic showing the sequence of HITI K with the template DNA comprising exons 45-55 as well as portions of intron 44 and intron 55 flanking the exons and comprising gRNA target sequences.

FIGS. 17A-17B. FIG. 17A shows ICE quantification of indels at the Runx1 locus and Psck9 locus in DNA amplified from cells nucleofected with Runx1 and Psck9 cutting controls. FIG. 17B shows deep amplicon sequencing quantification of modified alleles at the mdx locus in DNA amplified from cells nucleofected with the indicated Cas9 complexes.

FIGS. 18A-18D show results of a tamoxifen injection protocol. FIG. 18A-Schematic of tamoxifen injection protocol. FIG. 18B- Representative FACS analyses of satellite cells isolated at P21 from vehicle-injected Pax7-CreER^{T2+/-}; Rosa26-LSL-SpCas9-P2A-EGFP^{+/-} mice, or from three replicate Pax7-CreER^{T2+/-}; Rosa26-LSL-SpCas9-P2A-EGFP^{+/-} animals injected daily from P16-P19 and harvested 5 days after the first injection. FIG. 18C- Gating strategy for sorting EGFP- and EGFP+ cells from tamoxifen-treated Pax7-CreER^{T2+/-}; Rosa26-LSL-SpCas9-P2A-EGFP^{+/-} mice. Plot is pre-gated for satellite cell markers (CD45-Scal-Mac1-CXCR4+CD29+). FIG. 18D-Confocal images of satellite cells sorted from the EGFP- and EGFP+ gates in (C).

FIG. 19 shows immunofluorescence analysis of cross-sections of tibialis anterior muscle isolated at P21 from vehicle-injected or 5 dpi (P16-19) Pax7-CreER^{T2+/-}; Rosa26-LSL-SpCas9-P2A-EGFP^{+/-} mice. EGFP is detected in Pax7+ satellite cells of tamoxifen, but not vehicle, injected mice, and no EGFP is detected in muscle fibers.

FIGS. 20A-20B show results of a tamoxifen injection protocol. FIG. 20A-Schematic of tamoxifen injection protocol. FIG. 20B- Representative FACS analyses of satellite cells isolated at P42 from vehicle-injected Pax7-CreER^{T2+/-}; Rosa26-LSL-SpCas9-P2A-EGFP^{+/-} mice, or from three replicate Pax7-CreER^{T2+/-}; Rosa26-LSL-SpCas9-P2A-EGFP^{+/-} animals injected daily from P16-P19 and harvested 26 days after the first injection. Plots are pre-gated for satellite cell markers (CD45-Scal-Mac1-CXCR4+CD29+).

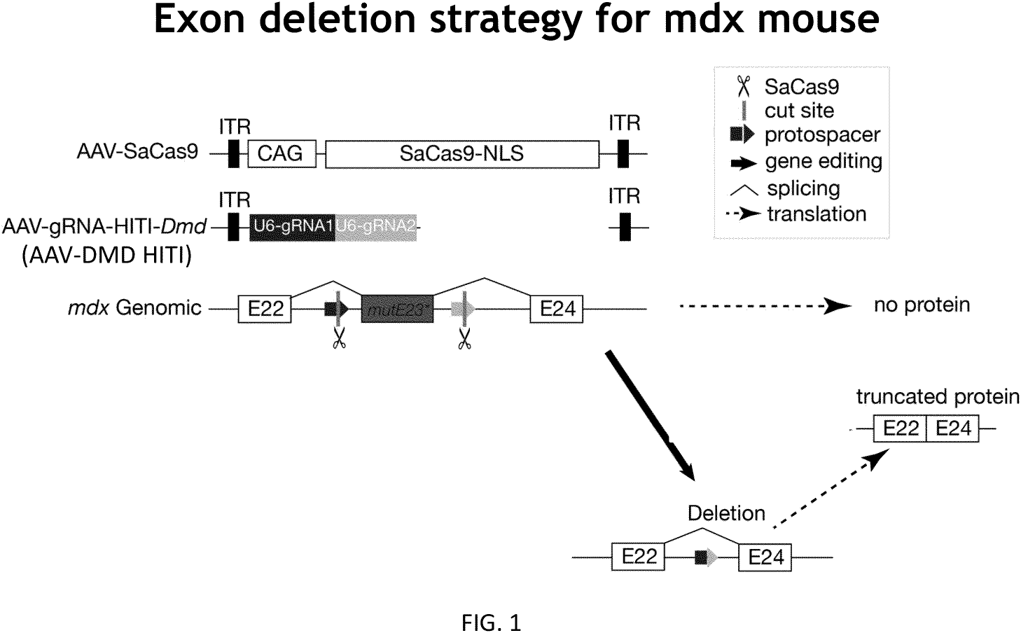

### DETAILED DESCRIPTION OF THE INVENTION

### Methods of Modifying Exons 45-55 of DMD

Some aspects of the present disclosure are related to a method for modifying the genome of a mammalian muscle or muscle precursor cell, comprising contacting the cell with a Cas protein and a first and second guide ribonucleic acid (gRNA), wherein the first gRNA hybridizes to a first target site located in intron 44 of DMD and the second gRNA hybridizes to a second target site located in intron 55 of DMD, thereby modifying the genome of the mammalian muscle or muscle precursor cell located between intron 44 and intron 55 of DMD.

As used herein, the phrase “muscle or muscle precursor cell” refers broadly to all classifications of muscle cells at all stages of development. Thus, “muscle or muscle precursor cell” encompasses both undifferentiated muscle cells, such as for example myoblasts, as well as differentiated muscle cells, such as for example terminally differentiated myotubes. “muscle or muscle precursor cell” also encompasses muscle cells of varying histological types, including but not limited to striated muscle cells (e.g., skeletal muscle cells), smooth muscle cells (e.g., intestinal muscle cells), and cardiac muscle cells. In some embodiments, a “muscle or muscle precursor cell” is a skeletal muscle cell or skeletal muscle precursor cell. In some embodiments, a “muscle or muscle precursor cell” is a skeletal muscle cell, skeletal muscle precursor cell, cardiac muscle cell, or cardiac muscle precursor cell.

The mammalian cell is not limited. In some embodiments, the cell is a cell of a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, and wolf. In some embodiments, the cell is a cell of a human or a dog.

In some embodiments, the cell is a stem cell or an induced pluripotent stem cell. In some embodiments, the induced pluripotent stem cell is derived from a cell of a subject with a muscular dystrophy. In some embodiments, the induced pluripotent stem cell is derived from a cell of a subject with a muscular dystrophy and having a mutation located between intron 44 and intron 55 of the DMD gene. In some embodiments, the mutation is a frame-shift mutation.

The Cas protein is not limited. The Clustered Regularly Interspaced Short Palindromic Repeats (CRISPR) Type II system is a bacterial adaptive immune system that has been modified for use as an RNA-guided endonuclease technology for genome engineering. The bacterial system comprises two endogenous bacterial RNAs called crRNA and tracrRNA and a CRISPR-associated (Cas) nuclease, e.g., Cas9. The tracrRNA has partial complementarity to the crRNA and forms a complex with it. The Cas protein is guided to the target sequence by the crRNA/tracrRNA complex, which forms an RNA/DNA hybrid between the crRNA sequence and the complementary sequence in the target. For use in genome modification, the crRNA and tracrRNA components are often combined into a single chimeric guide RNA (sgRNA or gRNA) in which the targeting specificity of the crRNA and the properties of the tracrRNA are combined into a single transcript that localizes the Cas protein to the target sequence so that the Cas protein can cleave the DNA. The gRNA often comprises an approximately 20 nucleotide guide sequence complementary or homologous to the desired target sequence followed by about 80 nt of hybrid crRNA/tracrRNA. One of ordinary skill in the art appreciates that the guide RNA need not be perfectly complementary or homologous to the target sequence. For example, in some embodiments it may have one or two mismatches. The genomic sequence which the gRNA hybridizes is typically flanked on one side by a Protospacer Adjacent Motif (PAM) sequence although one of ordinary skill in the art appreciates that certain Cas proteins may have a relaxed requirement for a PAM sequence. The PAM sequence is present in the genomic DNA but not in the gRNA sequence. The Cas protein will be directed to any DNA sequence with the correct target sequence and PAM sequence. The PAM sequence varies depending on the species of bacteria from which the Cas protein was derived. Specific examples of Cas proteins include Cas1, Cas2, Cas3, Cas4, Cas5, Cas6, Cas7, Cas8, Cas9 and Cas10. In some embodiments, the Cas protein comprises a Cas9 protein. For example, Cas9 from Streptococcus pyogenes (Sp), Neisseria meningitides, Staphylococcus aureus, Streptococcus thermophiles, or Treponema denticola may be used. The PAM sequences for these Cas9 proteins are NGG, NNNNGATT, NNAGAA, NAAAAC, respectively. In some embodiments, the Cas9 is from Staphylococcus aureus (saCas9). In some embodiments, the Cas9 is a small Cas9 ortholog from Staphylococcus auricularis (SauriCas9), which recognizes a simple NNGG PAM, displays high activity for genome editing, and is compact enough to be packaged into an AAV for genome editing. In some embodiments, the Cas protein is Campylobacter jejuni (CjCas9), Neisseria meningitidis Cas9 (NmeCas9), Cas12b (see, Strecker et al., Nat Commun. 2019 Jan 22;10(1):212), or CasX (see, Nature. 2019 Feb 4. pii: 10.1038/s41586-019-0908-x. doi: 10.1038/s41586-019-0908-x).

A number of engineered variants of the Cas proteins have been developed and may be used in certain embodiments. For example, engineered variants of Cas9 are known in the art. Furthermore, it will be understood that a biologically active fragment or variant can be used. Other variations include the use of hybrid site specific nucleases. For example, in CRISPR RNA-guided FokI nucleases (RFNs) the FokI nuclease domain is fused to the amino-terminal end of a catalytically inactive Cas9 protein (dCas9) protein. RFNs act as dimers and utilize two guide RNAs (Tsai, QS, et al., Nat Biotechnol. 2014; 32(6): 569- 576). Site-specific nucleases that produce a single-stranded DNA break are also of use for genome editing. Such nucleases, sometimes termed “nickases” can be generated by introducing a mutation (e.g., an alanine substitution) at key catalytic residues in one of the two nuclease domains of a site specific nuclease that comprises two nuclease domains (such as ZFNs, TALENs, and Cas proteins). Examples of such mutations include D10A, N863A, and H840A in SpCas9 or at homologous positions in other Cas9 proteins. A nick can stimulate HDR at low efficiency in some cell types. Two nickases, targeted to a pair of sequences that are near each other and on opposite strands can create a single-stranded break on each strand (“double nicking”), effectively generating a DSB, which can optionally be repaired by HDR using a donor DNA template (Ran, F. A. et al. Cell 154, 1380-1389 (2013). In some embodiments, the Cas protein is a SpCas9 variant. In some embodiments, the SpCas9 variant is a R661A/Q695A/Q926A triple variant or a N497A/R661A/Q695A/ Q926A quadruple variant. See Kleinstiver et al., “High-fidelity CRISPR-Cas9 nucleases with no detectable genome-wide off-target effects,” Nature, Vol. 529, pp. 490-495 (and supplementary materials)(2016); incorporated herein by reference in its entirety. In some embodiments, the Cas protein is C2c1, a class 2 type V-B CRISPR-Cas protein. See Yang et al., “PAM-Dependent Target DNA Recognition and Cleavage by C2c1 CRISPR-Cas Endonuclease,” Cell, Vol. 167, pp. 1814-1828 (2016); incorporated herein by reference in its entirety. In some embodiments, the Cas protein is one described in US 20160319260 “Engineered CRISPR-Cas9 nucleases with Altered PAM Specificity” incorporated herein by reference.

In some embodiments, the target sequences for the first and second gRNA are SEQ ID NO: 6 and SEQ ID NO: 7. In some embodiments, the target sequences for the first and second gRNA SEQ ID NO: 8 and SEQ ID NO: 9. In some embodiments, the first and second gRNA are any gRNA pair or pair of gRNA target sequences described herein. In some embodiments, the first and second gRNA have a pair of gRNA target sequences shown in Table 1.

The DMD gene is not limited. In some embodiments, the DMD gene is human DMD gene (Gene ID: 1756). Dystrophin (DMD) gene is the largest known gene. DMD spans 2.2 Mb of the X chromosome and encodes predominantly a 14-kb transcript derived from 79 exons. The full-length dystrophin protein, as expressed in skeletal muscle, smooth muscle, and cardiomyocytes, is 3685 amino acids and has a molecular weight of 427 kD. The severe Duchenne phenotype is generally associated with the loss of full length dystrophin protein from skeletal and cardiac muscle, which leads to debilitating muscle degeneration and, ultimately, heart failure. A large number of different DMD mutations have been described, many of them resulting in either the severe Duchenne Muscular Dystrophy or the milder Becker Muscular Dystrophy. The Leiden University Medical Center maintains a database of mutations in the DMD gene (www.dmd.nl), incorporated herein by reference.

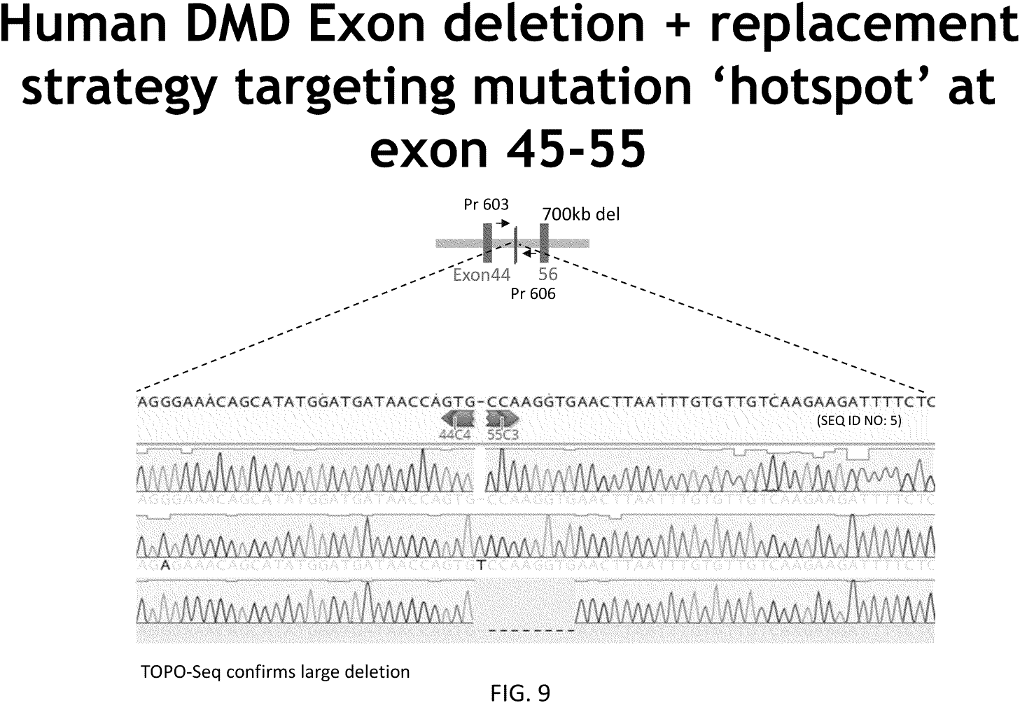

In some embodiments, the modification of the genome comprises a deletion of the nucleotide sequence between intron 44 and intron 55 of DMD. In some embodiments, the modified genome comprises the sequence of SEQ ID NO: 5 wherein the nucleotide signified as a “dash” in FIG. 9 is absent or comprises 1-10 inserted amino acids.

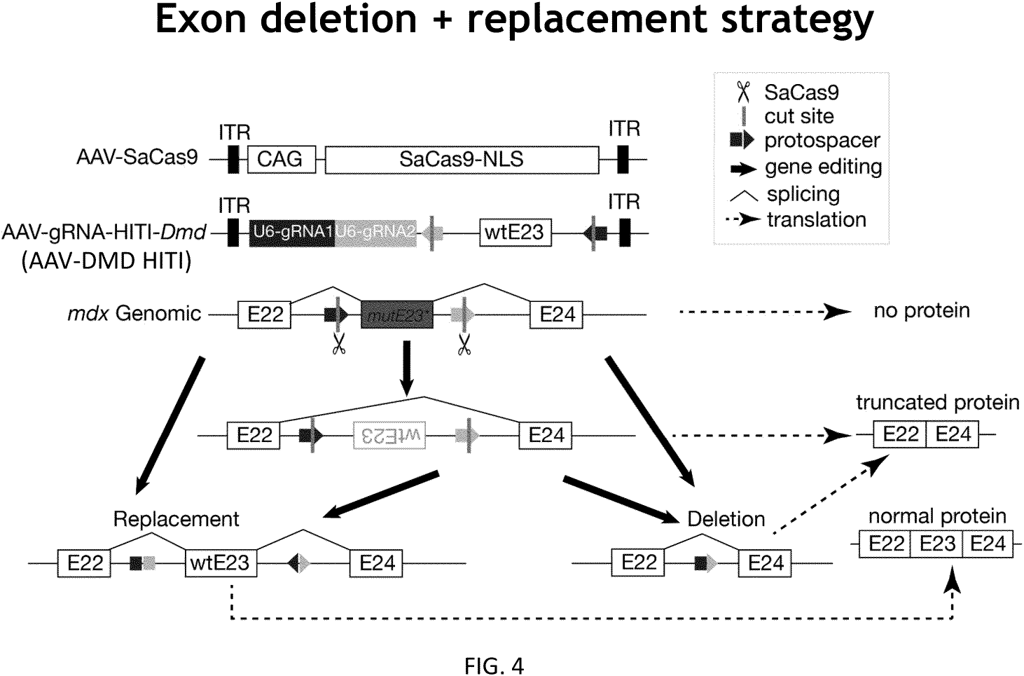

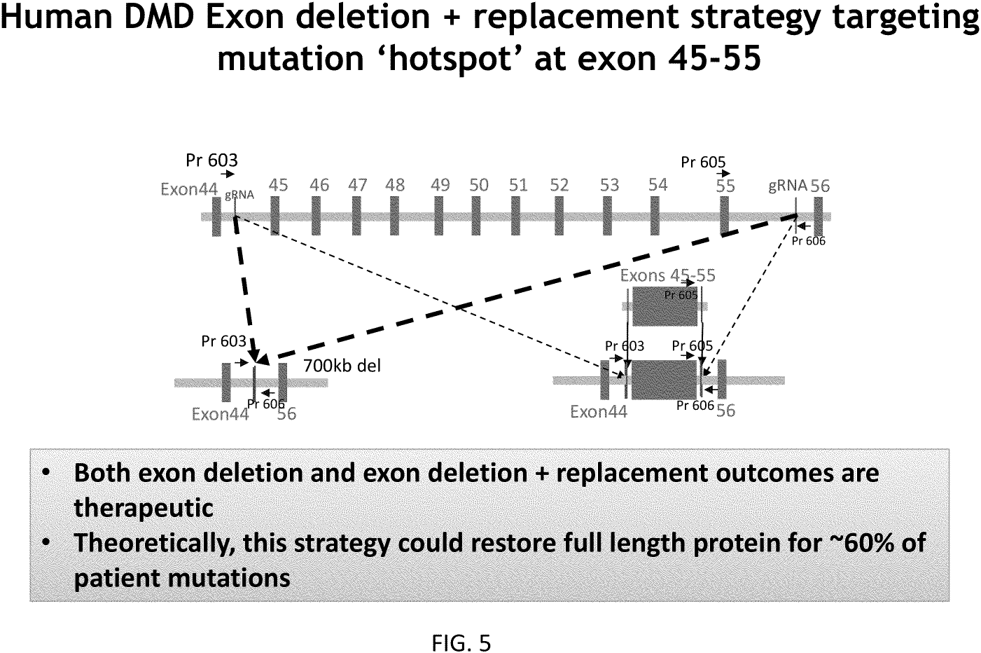

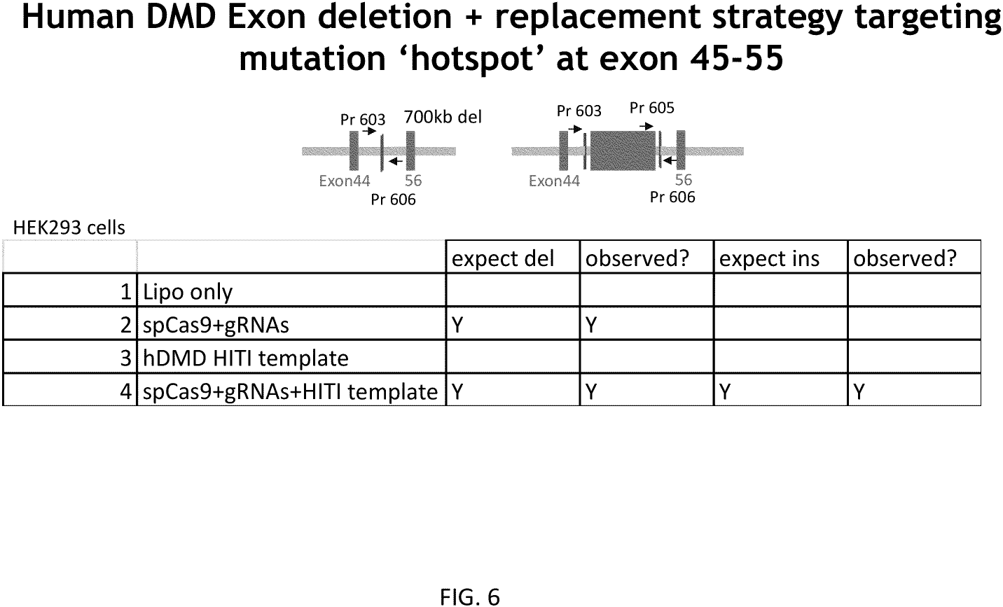

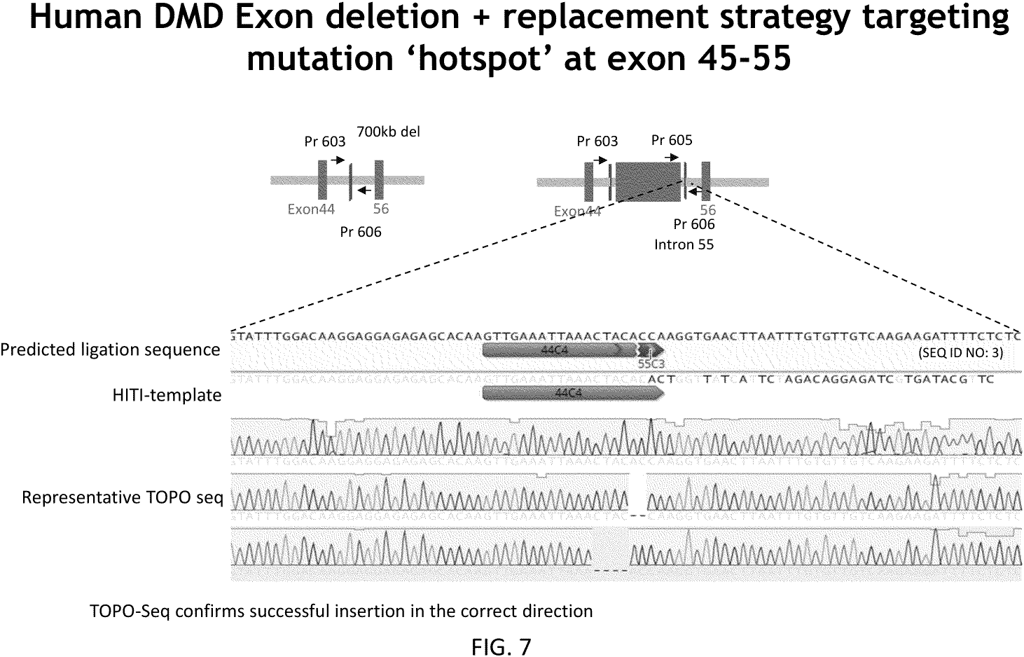

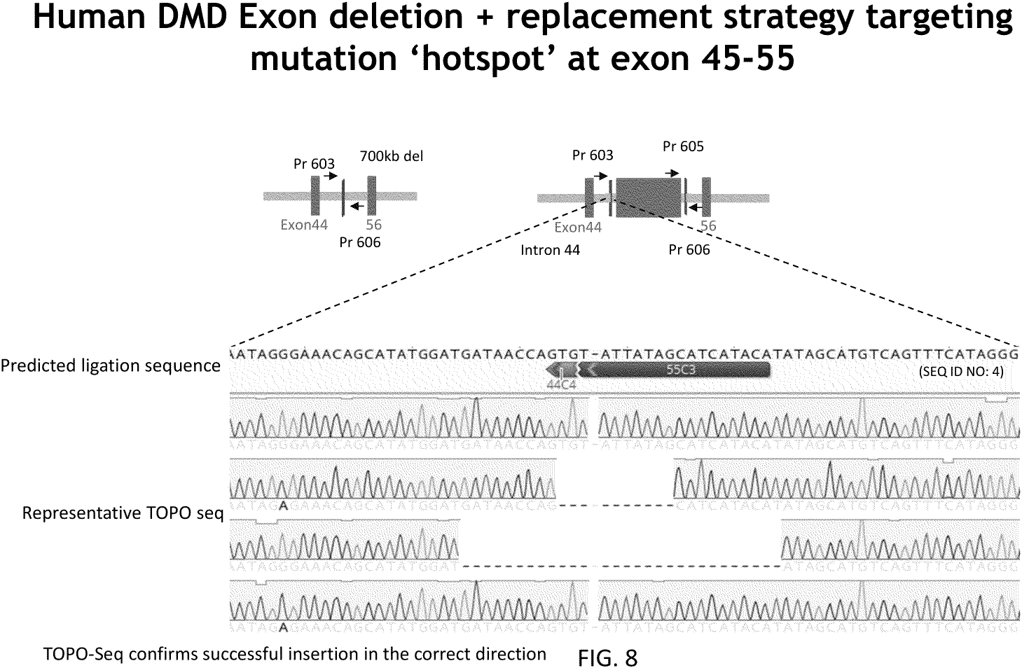

In some embodiments, the method further comprises a step of contacting the cell with template DNA comprising the nucleotide sequences of exons 45 to 55 of wild-type DMD. In some embodiments, the nucleotide sequence of exons 45-55 comprises or consists of a nucleotide sequence homologous or identical SEQ ID NO: 28 or a nucleotide sequence at least 85%, 90%, 91%, 92%, 93%, 94%, 95%, 96%, 97%, 98%, 99%, 99.5%, or 99.9% homologous to SEQ ID NO: 28. In some embodiments, the template sequence comprises or consists of a nucleotide sequence homologous or identical to SEQ ID NO: 10 or 11, or a portion thereof. In some embodiments, the template sequence is at least 80%, 85%, 90%, 91%, 92%, 93%, 94%, 95%, 96%, 97%, 98%, 99%, 99.5%, 99.9% or more homologous or identical to SEQ ID NO: 10, 11, or 28, or a portion thereof. In some embodiments, the template DNA comprises a portion of the first and second target sites flanking the nucleotide sequences of exons 45 to 55 of wild-type DMD. As used in this aspect, the template DNA comprises the sequence after cutting by Cas within the target sequences. The target sequences of the template DNA should be situated on the opposite side of the insert than the location of the target sites on either side of exons 45-55 in the genome of the cell. Thereby, upon insertion in the correct orientation of exons 45-55 from the template into the genomic DNA, the target sites will be disrupted. However, if exons 45-55 are inserted in the wrong orientation, the target sites will be reconstituted and may again be cut by the Cas protein upon hybridization with guide sequence. See, for example, FIG. 4.

In some embodiments, the modification of the genome comprises replacement of the nucleotide sequence between intron 44 and intron 55 of DMD with template DNA comprising exons 45 to 55 of wild-type DMD. In some embodiments, the replaced nucleotide sequence comprises or consists of SEQ ID NO: 28 or a nucleotide sequence at least 85%, 90%, 91%, 92%, 93%, 94%, 95%, 96%, 97%, 98%, 99%, 99.5%, or 99.9% homologous to SEQ ID NO: 28. In some embodiments, the replacement of the nucleotide sequence between intron 44 and intron 55 of DMD occurs via non-homologous end joining (NHEJ). Non-homologous end joining (NHEJ) is a pathway that repairs double-strand breaks in DNA. NHEJ is referred to as “non-homologous” because the break ends are directly ligated without the need for a homologous template, in contrast to homology directed repair, which requires a homologous sequence to guide repair.

In some embodiments, wherein the genome of the mammalian muscle or muscle precursor cell located between intron 44 and intron 55 of DMD comprises a mutation associated with a disease or condition. In some embodiments, the disease or condition is muscular dystrophy. In some embodiments, the muscular dystrophy is selected from myotonic muscular dystrophy, Duchenne muscular dystrophy (DMD), Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy. In some embodiments, the disease or condition is Duchenne muscular dystrophy.

In some embodiments, the cell is contacted with one or more viruses transducing one or more of the Cas protein, the first gRNA, the second gRNA, and/or the template DNA. Suitable viruses for use in the methods disclosed throughout the specification include, e.g., adenoviruses, adeno-associated viruses, retroviruses (e.g., lentiviruses), vaccinia virus and other poxviruses, herpesviruses (e.g., herpes simplex virus), and others. The virus may or may not contain sufficient viral genetic information for production of infectious virus when introduced into host cells, i.e., viral vectors may be replication-competent or replication-defective.

In some embodiments, the virus is adeno-associated virus. Adeno-associated virus (AAV) is a small (20 nm) replication-defective, nonenveloped virus. The AAV genome is a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA that inhibits ATPIF1, operably linked to a promoter, is inserted between the inverted terminal repeats (ITR) of the AAV genome. Adeno-associated viruses (AAV) and their use as vectors, e.g., for gene therapy, are also discussed in Snyder, RO and Moullier, P., Adeno-Associated Virus Methods and Protocols, Methods in Molecular Biology, Vol. 807. Humana Press, 2011.

In some embodiments, the AAV is AAV serotype 6, 8, 9, 10 or Anc80 (disclosed in WO2015054653, incorporated herein by reference). In some embodiments, the AAV serotype is AAV serotype 2. Any AAV serotype, or modified AAV serotype, may be used as appropriate and is not limited.

Another suitable AAV may be, e.g., rhlO [see, e.g., WO 2003/042397]. Still other AAV sources may include, e.g., AAV9 [see, e.g., US 7,906,111; US 2011-0236353-A1], and/or hu37 [see, e.g., US 7,906,111; US 2011-0236353-A1], AAV1, AAV2, AAV3, AAV4, AAV5, AAV6, AAV6.2, AAV7, AAV8, [see, e.g., U.S. Pat. 7790449; U.S. Pat. 7282199] and others. See, e.g., WO 2003/042397; WO 2005/033321, WO 2006/110689; U.S. Pat. 7790449; U.S. Pat. 7282199; and US 7588772 B2 for sequences of these and other suitable AAV, as well as for methods for generating AAV vectors. Still other AAV may be selected, optionally taking into consideration tissue preferences of the selected AAV capsid. A recombinant AAV vector (AAV viral particle) may comprise, packaged within an AAV capsid, a nucleic acid molecule containing a 5 ′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3 ′ ITR. A shortened version of the 5′ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self- complementary recombinant adeno-associated virus (scAAV) vectors promote efficient transduction independently of DNA synthesis”, Gene Therapy, (August 2001), Vol 8, Number 16, Pages 1248- 1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

A single-stranded AAV viral vector may be used. Methods for generating and isolating AAV viral vectors suitable for delivery to a subject are known in the art. See, e.g., U.S. Pat. 7790449; U.S. Pat. 7282199; WO 2003/042397; WO 2005/033321, WO 2006/110689; and US 7588772 B2. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV - the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus- adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. Pats., the contents of which is incorporated herein by reference in its entirety: 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless” -containing only the transgene of interest flanked by the signals required for amplification and packaging of the artificial genome), but these genes may be supplied during production.

The one or more viruses may contain a promoter capable of directing expression (e.g., expression of a Cas protein, template DNA, and/or one or more gRNAs) in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, the promoter is selected from a CMV promoter, U6 promoter, an H1 promoter, a constitutive promoter, and a ubiquitous promoter. In some embodiments, the promoter directs expression in a particular cell type. For example, a muscle precursor cell specific promoter.

In some embodiments of each of the methods disclosed herein, a suitable tissue specific promoter can be obtained by a person of ordinary skill in the art from the tissue specific promoters set forth in “TiProD: Tissue specific promoter Database” available on the world-wide web at tiprod.bioinf.med.uni-goettingen.de/.

In some embodiments, the cell is contacted with a first virus transducing a nucleic acid encoding the Cas protein in the cell and a second virus transducing a nucleic acid encoding the first gRNA, the second gRNA, and the template DNA in the cell. In some embodiments, at least the first virus or second virus is an AAV virus. In some embodiments, the cell is contacted with a first virus transducing a nucleic acid encoding the Cas protein in the cell and a second virus transducing a nucleic acid of SEQ ID NO: 26 (HITI B). In some embodiments, the cell is contacted with a first virus transducing a nucleic acid encoding the Cas protein in the cell and a second virus transducing a nucleic acid of SEQ ID NO: 27 (HITI K).

In some embodiments, the treated cell with a modified genome expresses functional truncated dystrophin lacking an amino acid sequence coded by exons 45 to 55 of the DMD gene. In some embodiments, the treated cell with a modified genome expresses full length dystrophin comprising amino acid sequences coded by exons 45 to 55 of wild-type DMD. In some embodiments,, the treated cell with a modified genome expresses full length dystrophin comprising amino acid sequences coded by SEQ ID NO: 28 or a nucleotide sequence at least 85%, 90%, 91%, 92%, 93%, 94%, 95%, 96%, 97%, 98%, 99%, 99.5%, or 99.9% homologous to SEQ ID NO: 28.

In some embodiments, the cell is contacted in vitro. In some embodiments, the cell is obtained from a subject and contacted ex vivo. In some embodiments, the treated cell is administered to the subject. In some embodiments, the cell is contacted in vivo. In some embodiments, a subject is administered one or more viruses described herein resulting in contact of the cell with Cas9, gRNAs and template DNA described herein.

### Methods of Treating Muscular Dystrophy Patient Having a Mutation Located Between Intron 44 and Intron 55

Some aspects of the present disclosure are directed to a method of treating a muscular dystrophy in a subject in need thereof, comprising contacting a muscle or muscle precursor cell of the patient with a Cas protein and a first and second guide ribonucleic acid (gRNA), wherein the first gRNA hybridizes to a first target site located in intron 44 of DMD and the second gRNA hybridizes to a second target site located in intron 55 of DMD, thereby modifying the genome of the cell, wherein the subject’s genome comprises a mutation located between intron 44 and intron 55 of the DMD gene.

In some embodiments, at least 1%, 2%, 3%, 5%, 10%, 15%, 20%, 25%, 30%, 40%, 50%, 60%, 70%, 80%, 90%, 95%, or more of the subject’s muscle or muscle precursor cells have their genomes modified by the methods disclosed herein.

As used herein, “treat,” “treatment,” “treating,” or “amelioration” when used in reference to a disease, disorder or medical condition (e.g., muscular dystrophy), refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state as compared to that expected in the absence of treatment.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

The muscular dystrophy is not limited and may be any muscular dystrophy described herein. In some embodiments, the muscular dystrophy is Becker muscular dystrophy or Duchenne muscular dystrophy. The muscle or muscle precursor cell of the patient is not limited and may be any muscle or muscle precursor cell described herein. In some embodiments, the cell is a human cell or a canine cell. In some embodiments, the muscle or muscle precursor cell is a skeletal muscle cell or skeletal muscle precursor cell.

The Cas protein is not limited and may be any Cas protein described herein. In some embodiments, the Cas protein is Cas9 (e.g., saCas9). The gRNAs are not limited and may be any suitable gRNA. In some embodiments, the target sequence for the gRNA are selected from target sequences provided in SEQ ID NOS: 6-9. In some embodiments, the gRNA pairs bind to target sequences provided in SEQ ID NOS: 6-7 or SEQ ID NOS: 8-9. In some embodiments, the gRNA pairs bind to target sequences provided in Table 1.

In some embodiments, the modification of the genome comprises a deletion of the nucleotide sequence between intron 44 and intron 55 of DMD.

In some embodiments, the method further comprises contacting the cell with template DNA comprising the nucleotide sequences of exons 45 to 55 of wild-type DMD. In some embodiments, the nucleotide sequences of exons 45 to 55 comprises or consists of SEQ ID NO: 28. In some embodiments, the template DNA comprises a portion of the first and second target sites flanking the nucleotide sequences of exons 45 to 55 of wild-type DMD. As described herein, the target sites may be located at the opposite ends of the nucleotide sequences of exons 45 to 55 of wild-type DMD than the location of the target sequences in the genome of the subject. Such configuration results in the loss of the targeting sequence upon correct insertion of the substituted exon sequences but reconstitution of the target sequences (possibly followed by cleavage by Cas protein) if the substituted exon sequences are not inserted in the correct orientation.

In some embodiments, the modification of the genome comprises replacement of the nucleotide sequence between intron 44 and intron 55 of DMD with template DNA comprising exons 45 to 55 of wild-type DMD. In some embodiments, the modification of the genome comprises replacement of the nucleotide sequence between intron 44 and intron 55 of DMD with a nucleotide sequence of SEQ ID NO: 10 or 11. In some embodiments, the replacement of the nucleotide sequence between intron 44 and intron 55 of DMD occurs via non-homologous end joining (NHEJ).

In some embodiments, the subject is administered one or more viruses transducing the Cas protein, the first gRNA, the second gRNA, or the template DNA. The viruses are not limited and may be any virus described herein. In some embodiments, the one or more viruses are AAV viruses. In some embodiments, the subject is administered a first virus transducing a nucleic acid encoding the Cas protein in the cell and a second virus transducing a nucleic acid encoding the first gRNA, the second gRNA, and the template DNA in the cell. In some embodiments, the second virus transduces a nucleotide sequence of SEQ ID NO: 26 or 27.

In some embodiments, the cell with a modified genome expresses functional truncated dystrophin lacking an amino acid sequence coded by exons 45 to 55 of wild-type DMD. In some embodiments, the cell with a modified genome expresses full length dystrophin comprising amino acid sequences coded by exons 45 to 55 of wild-type DMD. In some embodiments, the cell with a modified genome expresses full length dystrophin comprising amino acid sequences coded by SEQ ID NO: 28 or a nucleotide sequence at least 85%, 90%, 91%, 92%, 93%, 94%, 95%, 96%, 97%, 98%, 99%, 99.5%, or 99.9% homologous to SEQ ID NO: 28.

In some embodiments, the cell is contacted ex vivo or in vivo. As used herein, “contacting” a cell with one or more viruses can comprise administration of the virus systemically (e.g., intravenously) or locally (e.g., intramuscular injection) into the subject. Alternatively, other routes of administration may be selected (e.g., oral, inhalation, intranasal, intratracheal, intraarterial, intraocular, intravenous, intramuscular, and other parental routes). The method of contacting is not limited and may be any suitable method available in the art.

In some embodiments, virus compositions can be formulated in dosage units to contain an amount of replication-defective virus that is in the range of about 1.0 x 10⁹ GC to about 1.0 x 10¹⁵ GC (to treat an average subject of 70 kg in body weight), and preferably 1.0 x 10¹² GC to 1.0 x 10¹⁴ GC for a human patient. Preferably, the dose of replication-defective virus in the formulation is 1.0 x 10⁹ GC, 5.0 X 10⁹ GC, 1.0 X 10¹⁰ GC, 5.0 X 10¹⁰ GC, 1.0 X 10¹¹ GC, 5.0 X 10¹¹ GC, 1.0 X 10¹² GC, 5.0 X 10¹² GC, or 1.0 x 10¹³ GC, 5.0 X 10¹³ GC, 1.0 X 10¹⁴ GC, 5.0 X 10¹⁴ GC, or 1.0 x 10¹⁵ GC.

### Further Methods of Treating Muscular Dystrophy

Some aspects of the present disclosure are directed to a method for modifying the genome of a mammalian muscle or muscle precursor cell, comprising contacting the cell with a Cas protein and a first and second guide ribonucleic acid (gRNA), wherein the first gRNA hybridizes to a first target site located in a first intron of DMD and the second gRNA hybridizes to a second target site located in another intron of DMD, thereby modifying the genome of the mammalian muscle or muscle precursor cell located between the first and second target sites.

The DMD exons located between the target sites are not limited. In some embodiments, the DMD exons located between the target sites are not necessary for producing a functional dystrophin protein (i.e., a functional truncated dystrophin protein). In some embodiments, DMD exon 23 is located between the first and second target sites.

In some embodiments, at least 1%, 2%, 3%, 5%, 10%, 15%, 20%, 25%, 30%, 40%, 50%, or more of the subject’s muscle or muscle precursor cells have their genomes modified by the methods disclosed herein.

The muscular dystrophy is not limited and may be any muscular dystrophy described herein. In some embodiments, the muscular dystrophy is Becker muscular dystrophy or Duchenne muscular dystrophy. The muscle or muscle precursor cell of the patient is not limited and may be any muscle or muscle precursor cell described herein. In some embodiments, the cell is a human cell. In some embodiments, the muscle or muscle precursor cell is a skeletal muscle cell or skeletal muscle precursor cell. In some embodiments, the cell is a stem cell or an induced pluripotent stem cell. In some embodiments, the induced pluripotent stem cell is derived from a cell of the subject.

The Cas protein is not limited and may be any Cas protein described herein. In some embodiments, the Cas protein is Cas9 (e.g., saCas9, spCas9, SauriCas9, KKHCas9). In some embodiments, the Cas protein is sufficiently small to be packaged in a suitable viral vector (e.g., AAV) either alone or with one to two gRNAs. The gRNAs are not limited and may be any suitable gRNA.

In some embodiments, the method further comprises contacting the cell with template DNA comprising the nucleotide sequences of DMD exons located between the first and second target sites. In some embodiments, the template DNA comprises a portion of the first and second target sites flanking the nucleotide sequences of DMD exons located between the first and second target sites. As described herein, the target sites may be located at the opposite ends of the nucleotide sequences of the DMD exons than the location of the target sequences in the genome of the subject. Such configuration results in the loss of the targeting sequence upon correct insertion of the substituted exon sequences but reconstitution of the target sequences (possibly followed by cleavage by Cas protein) if the substituted exon sequences are not inserted in the correct orientation.

In some embodiments, the modification of the genome comprises replacement of the nucleotide sequence located between the first and second target sites with template DNA comprising the nucleotide sequences of DMD exons located between the first and second target sites. In some embodiments, replacement of the nucleotide sequence with template sequence occurs via non-homologous end joining (NHEJ).

In some embodiments, the genome of the cell located between the first and second target sites comprises a mutation associated with a disease or condition. The disease or condition is not limited and may be any disease or condition described herein. In some embodiments, the disease or condition is a muscular dystrophy. In some embodiments, the disease or condition is Duchenne muscular dystrophy or Becker muscular dystrophy.

In some embodiments, the cell is contacted with one or more viruses transducing the Cas protein, the first gRNA, the second gRNA, or the template DNA. In some embodiments, the cell is contacted with a first virus transducing a nucleic acid encoding the Cas protein in the cell and a second virus transducing a nucleic acid encoding the first gRNA, the second gRNA, and the template DNA in the cell. The viruses are not limited and may be any virus described herein. In some embodiments, at least the first virus or second virus is an AAV virus.

In some embodiments, the cell with a modified genome expresses functional truncated dystrophin lacking an amino acid sequence coded by one or more DMD exons. In some embodiments, the cell with a modified genome expresses full length dystrophin.

In some embodiments, the cell is contacted in vitro, ex vivo, or in vivo.

### Compositions

Some aspects of the present disclosure are related to a composition comprising a first virus transducing a nucleic acid encoding a Cas protein in a cell and a second virus transducing a nucleic acid encoding a first gRNA, a second gRNA, and a template DNA in a cell, wherein the first gRNA hybridizes to a first target site located in a first intron of DMD and the second gRNA hybridizes to a second target site located in another intron of DMD, and wherein the template DNA codes for one or more DMD exons located between the first and second targets sites. In some embodiments, the Cas protein is a Cas protein described herein (e.g., Cas9). In some embodiments, the first gRNA, second gRNA, and template DNA are described herein. In some embodiments, the composition comprising a first AAV and second AAV can be used to replace a sequence of DMD in a subject located between introns 44 and 55 of DMD with a sequence coding for exons 45-55. In some embodiments, the first AAV and second AAV for use in replacing a sequence of DMD in a subject located between introns 44 and 55 of DMD with a sequence coding for exons 45-55 is described herein. In some embodiments, the composition comprising a first AAV and second AAV can be used to replace a sequence of DMD in a subject located between introns 22 and 23 of DMD with a sequence coding for exon 23.

The terms “decrease,” “reduce,” “reduced,” “reduction,” “decrease,” and “‘inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a “p” value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); RobertS. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

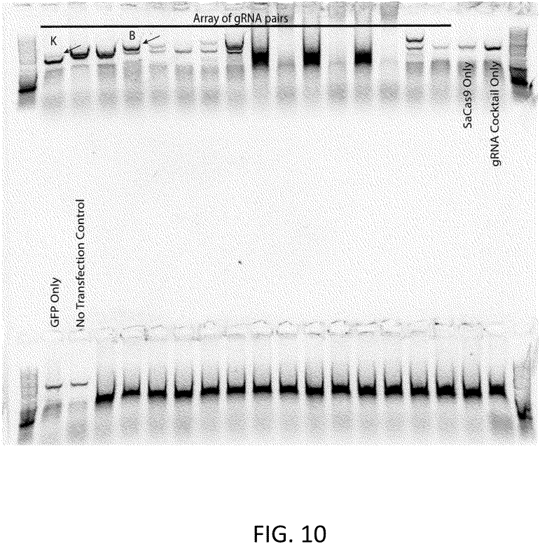

Sixteen candidate Staphylococcus aureus Cas9 (SaCas9) compatible gRNA pairs targeting separate regions within intron 44 or intron 55 of the human DMD gene were identified. A combinatorial approach was then used to test their cutting efficiency in HEK293T cells. Specifically, cells were transfected with SaCas9 and gRNAs, and DNA was extracted from the transfected cells 5 days post-transfection. For each gRNA pair, primers upstream and downstream of the most distal cut sites were designed and used to PCR amplify the junction between introns 44 and 55, which would amplify by PCR only if each gRNA cut efficiently and non-homologous end joining (NHEJ) ligated the two junctions. The amplified products were run on an agarose gel and screened for the expected size. Guide pairs “B” and “K” were identified as the most efficient, based on expected size and band strength (FIG. 10).

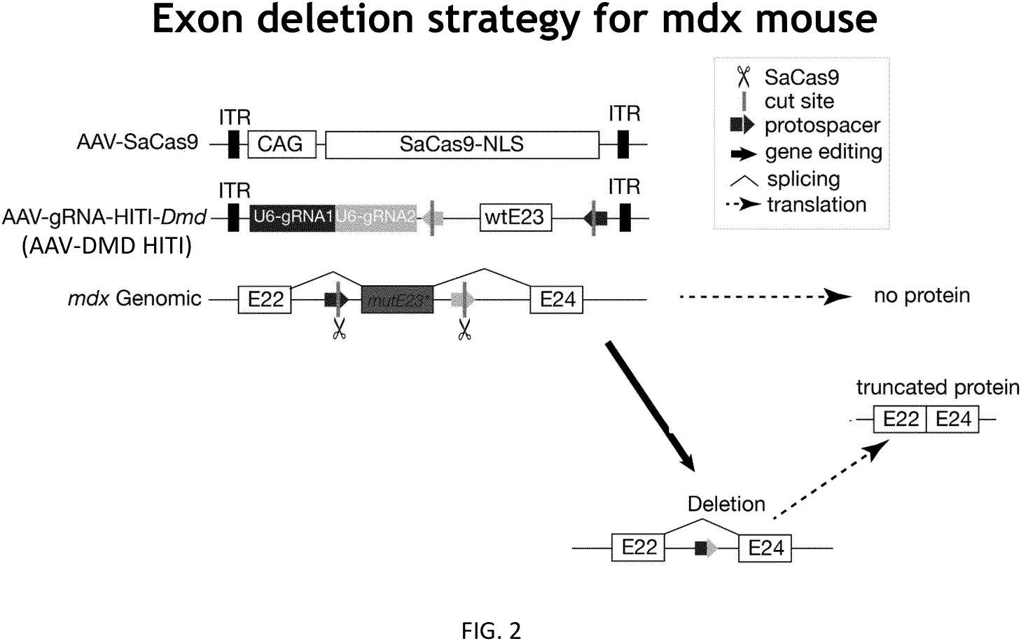

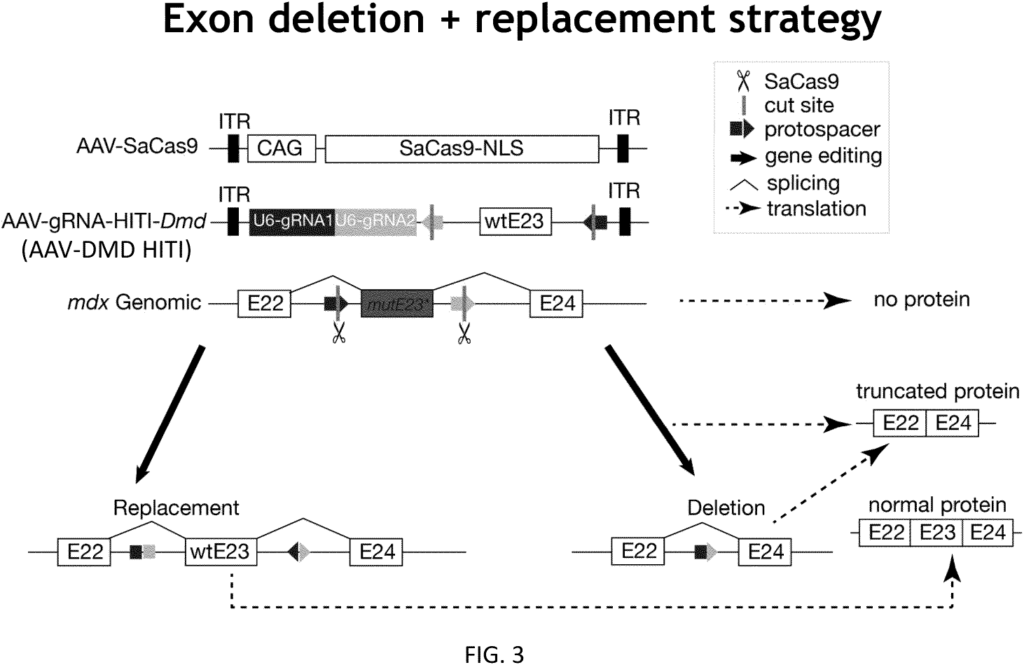

To test whether a double-cut-and-replace strategy could be used to replace exons 45-55 at the genomic level, two templates each containing the correct coding sequence of exons 45-55 as well as approximately 200 bp of the flanking intronic sequence on either end, were designed. Each template was also flanked by one of the guide pairs in the reverse direction (i.e. reverse of intron 55 gRNA proximal to exon 45 and reverse of intron 44 gRNA proximal to exon 55). See, for example, FIG. 2 showing gRNA1 cut sites in the blue region of the AAV-gRNA-HITI-Dmd template and blue region of mdx genome, as well as gRNA2 cut sites in the yellow region of the template and mdx genome.

To verify that the template, through NHEJ, could accurately be inserted between the genomic cut sites, each guide pair, along with their corresponding DNA template, were separately transfected into HEK293T cells and DNA was harvested 5 days post-transfection. In order to detect accurate insertion of the template, PCR for the presence of a junction between the genomic intron 44 and exon 45 on the template as well as the junction between exon 55 on the template and the genomic intron 55 was performed. Additionally, intron-intron junction without template insertion were assayed for, since this possibility would also be therapeutic.

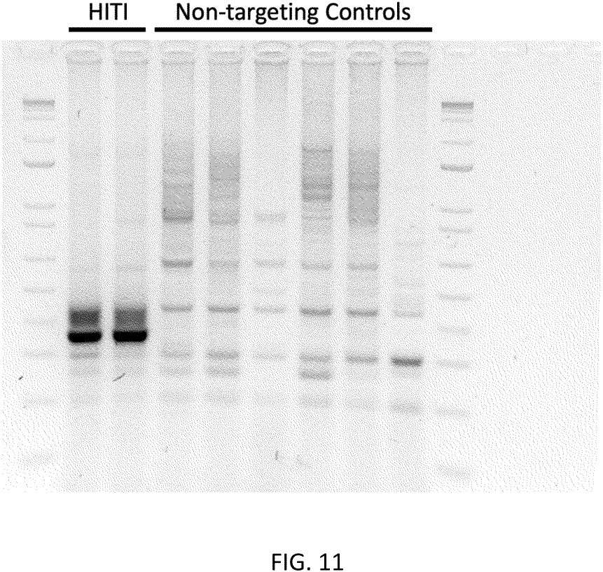

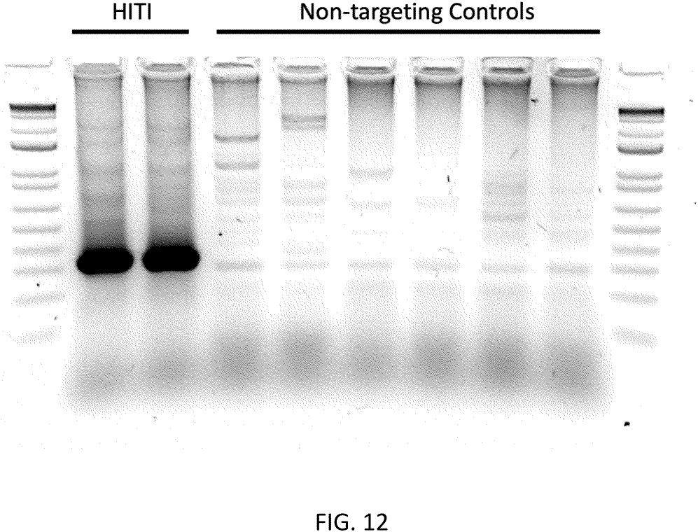

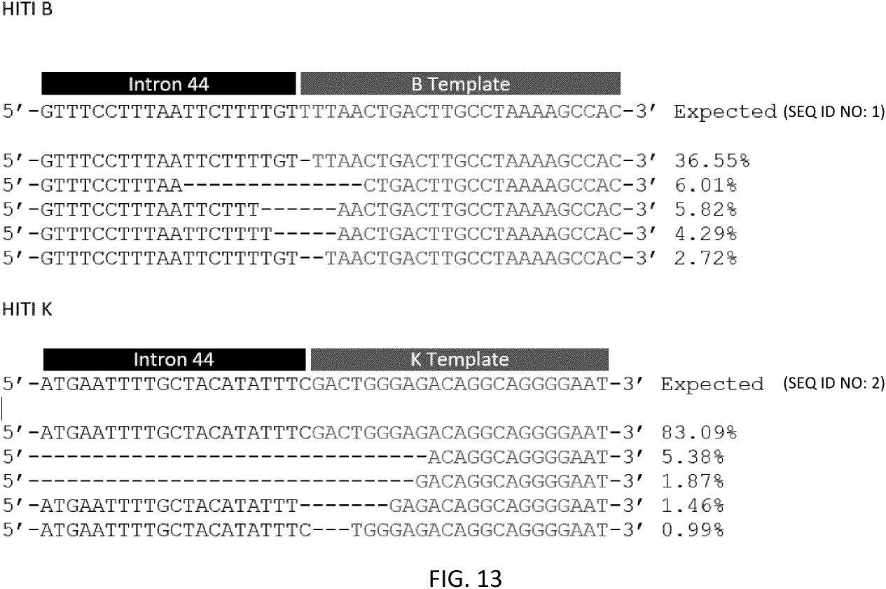

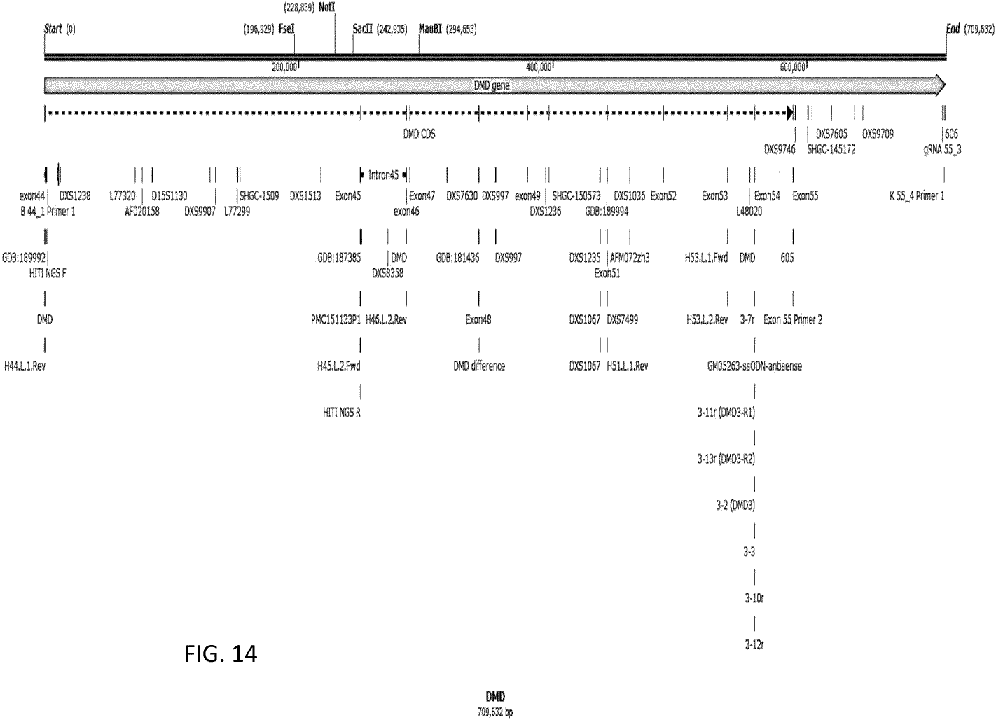

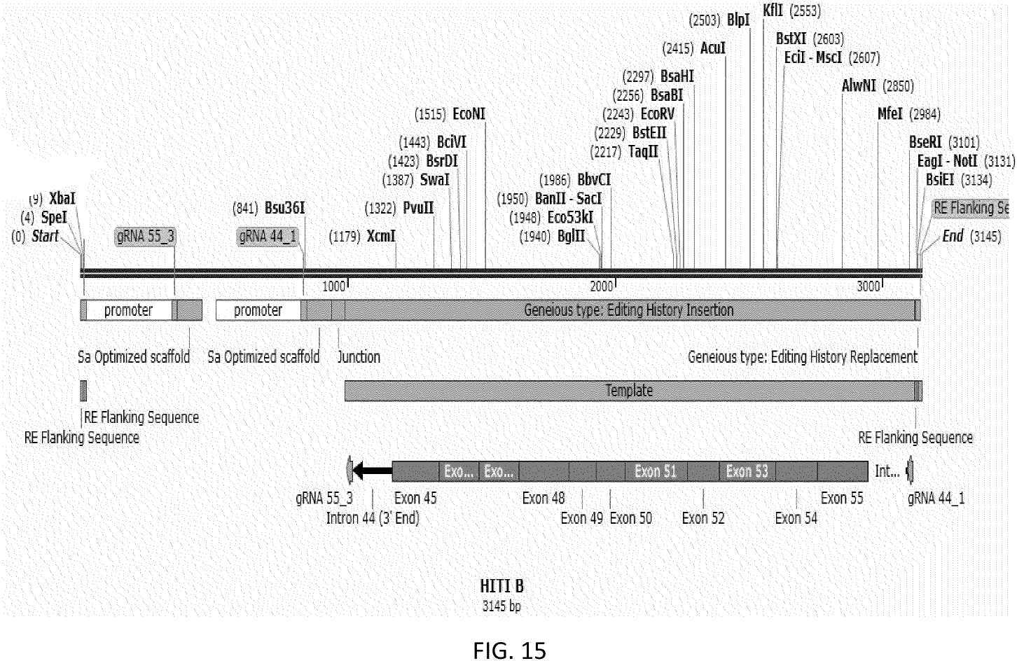

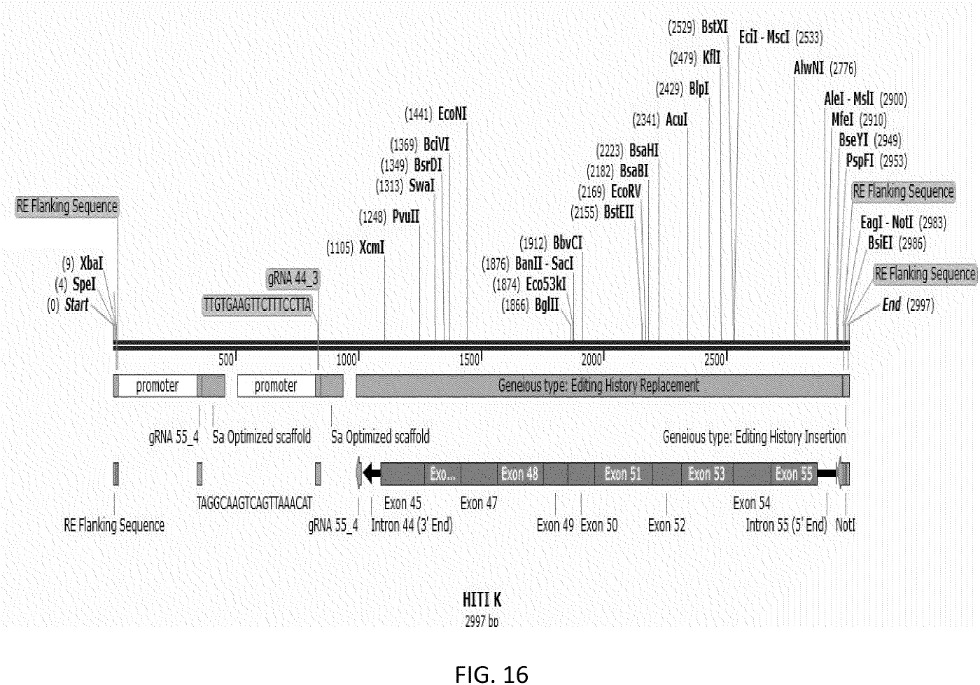

A band at the expected size was found when following amplification for the junction between exon 55 on the template and intron 55 for both guide pairs (FIG. 11) as well as a band at the expected size for the intron-intron junction sans template for both guide pairs (FIG. 12). To quantify the frequency of ligation events and screen for indels, deep amplicon sequencing was performed on the junction between intron 44 and exon 45 on the template (FIG. 13). Interestingly, a difference in efficiency of -1 or perfect ligations between the two guide pairs (“B” 37%, “K” 83%) was detected. This may be due to microhomology effects, or due to guide pair “B” having a larger number of off-target sites.

Following these experiments, follow-up experiments have been pursued to further characterize the system and its effects. In order to accurately quantify RNA and protein production following correction, the previous transfections with human DMD myoblasts containing mutations within the span of exons 45 to 55 which lead to a loss of dystrophin will be repeated. This experiment will allow detection of whether DNA-level edits lead to a rescue in RNA and protein production of dystrophin. The next step will be to move in vivo by using a human xenograft model, where human muscle is engrafted in mice such that AAV vectors carrying the above-described genome editing machinery can be injected systemically into the mice to transduce the human muscle.

### Sequences

gRNA and Template Sequences

HITI B gRNAs target sequences: CTAAGGAAAGAACTTCACAA (SEQ ID NO: 6) and TTGTGAAGTTCTTTCCTTAG (SEQ ID NO: 7) (i.e., gRNA pair “B”)

HITI K gRNAs: CTGCCTGTCTCCCAGTCAAA (SEQ ID NO: 8) and ATTTTGCTACATATTTCAGG (SEQ ID NO: 9) (i.e., gRNA pair “K”)

HITI B Template

(SEQ ID NO: 10):ACTCGGATGTTTAACTGACTTGCCTACATGTCAG
TTTCATAGGGAAATTTTCACATGGAGCTTTTGTATTTCTTTCTTTGCCAG
TACAACTGCATGTGGTAGCACACTGTTTAATCTTTTCTCAAATAAAAAGA
CATGGGGCTTCATTTTTGTTTTGCCTTTTTGGTATCTTACAGGAACTCCA
GGATGGCATTGGGCAGCGGCAAACTGTTGTCAGAACATTGAATGCAACTG
GGGAAGAAATAATTCAGCAATCCTCAAAAACAGATGCCAGTATTCTACAG
GAAAAATTGGGAAGCCTGAATCTGCGGTGGCAGGAGGTCTGCAAACAGCT
GTCAGACAGAAAAAAGAGGCTAGAAGAACAAAAGAATATCTTGTCAGAAT
TTCAAAGAGATTTAAATGAATTTGTTTTATGGTTGGAGGAAGCAGATAAC
ATTGCTAGTATCCCACTTGAACCTGGAAAAGAGCAGCAACTAAAAGAAAA
GCTTGAGCAAGTCAAGTTACTGGTGGAAGAGTTGCCCCTGCGCCAGGGAA
TTCTCAAACAATTAAATGAAACTGGAGGACCCGTGCTTGTAAGTGCTCCC
ATAAGCCCAGAAGAGCAAGATAAACTTGAAAATAAGCTCAAGCAGACAAA
TCTCCAGTGGATAAAGGTTTCCAGAGCTTTACCTGAGAAACAAGGAGAAA
TTGAAGCTCAAATAAAAGACCTTGGGCAGCTTGAAAAAAAGCTTGAAGAC
CTTGAAGAGCAGTTAAATCATCTGCTGCTGTGGTTATCTCCTATTAGGAA
TCAGTTGGAAATTTATAACCAACCAAACCAAGAAGGACCATTTGACGTTA
AGGAAACTGAAATAGCAGTTCAAGCTAAACAACCGGATGTGGAAGAGATT
TTGTCTAAAGGGCAGCATTTGTACAAGGAAAAACCAGCCACTCAGCCAGT
GAAGAGGAAGTTAGAAGATCTGAGCTCTGAGTGGAAGGCGGTAAACCGTT
TACTTCAAGAGCTGAGGGCAAAGCAGCCTGACCTAGCTCCTGGACTGACC
ACTATTGGAGCCTCTCCTACTCAGACTGTTACTCTGGTGACACAACCTGT
GGTTACTAAGGAAACTGCCATCTCCAAACTAGAAATGCCATCTTCCTTGA
TGTTGGAGGTACCTGCTCTGGCAGATTTCAACCGGGCTTGGACAGAACTT
ACCGACTGGCTTTCTCTGCTTGATCAAGTTATAAAATCACAGAGGGTGAT
GGTGGGTGACCTTGAGGATATCAACGAGATGATCATCAAGCAGAAGGCAA
CAATGCAGGATTTGGAACAGAGGCGTCCCCAGTTGGAAGAACTCATTACC
GCTGCCCAAAATTTGAAAAACAAGACCAGCAATCAAGAGGCTAGAACAAT
CATTACGGATCGAATTGAAAGAATTCAGAATCAGTGGGATGAAGTACAAG
AACACCTTCAGAACCGGAGGCAACAGTTGAATGAAATGTTAAAGGATTCA
ACACAATGGCTGGAAGCTAAGGAAGAAGCTGAGCAGGTCTTAGGACAGGC
CAGAGCCAAGCTTGAGTCATGGAAGGAGGGTCCCTATACAGTAGATGCAA
TCCAAAAGAAAATCACAGAAACCAAGCAGTTGGCCAAAGACCTCCGCCAG
TGGCAGACAAATGTAGATGTGGCAAATGACTTGGCCCTGAAACTTCTCCG
GGATTATTCTGCAGATGATACCAGAAAAGTCCACATGATAACAGAGAATA
TCAATGCCTCTTGGAGAAGCATTCATAAAAGGGTGAGTGAGCGAGAGGCT
GCTTTGGAAGAAACTCATAGATTACTGCAACAGTTCCCCCTGGACCTGGA
AAAGTTTCTTGCCTGGCTTACAGAAGCTGAAACAACTGCCAATGTCCTAC
AGGATGCTACCCGTAAGGAAAGGCTCCTAGAAGACTCCAAGGGAGTAAAA
GAGCTGATGAAACAATGGCAAGTAAGTCAGGCATTTCCGCTTTAGCACTC
TTGTGGATCCAATTGAACAATTCTCAGCATTTGTACTTGTAACTGACAAG
CCAGGGACAAAACAAAATAGTTGCTTTTATACAGCCTGATGTATTTCGGT
ATTTGGACAAGGAGGAGAGAGCTAAGGAAAGAACTTCACAAAAGAAT

HITI K Template

(SEQ ID NO: 11):GAACTCCAGGATGGCATTGGGCAGCGGCAAACTG
TTGTCAGAACATTGAATGCAACTGGGGAAGAAATAATTCAGCAATCCTCA
AAAACAGATGCCAGTATTCTACAGGAAAAATTGGGAAGCCTGAATCTGCG
GTGGCAGGAGGTCTGCAAACAGCTGTCAGACAGAAAAAAGAGGCTAGAAG
AACAAAAGAATATCTTGTCAGAATTTCAAAGAGATTTAAATGAATTTGTT
TTATGGTTGGAGGAAGCAGATAACATTGCTAGTATCCCACTTGAACCTGG
AAAAGAGCAGCAACTAAAAGAAAAGCTTGAGCAAGTCAAGTTACTGGTGG
AAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACT
TGAAAATAAGCTCAAGCAGACAAATCTCCAGTGGATAAAGGTTTCCAGAG
CTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCT
GCTGTGGTTATCTCCTATTAGGAATCAGTTGGAAATTTATAACCAACCAA
ACCAAGAAGGACCATTTGACGTTAAGGAAACTGAAATAGCAGTTCAAGCT
AAACAACCGGATGTGGAAGAGATTTTGTCTAAAGGGCAGCATTTGTACAA
GGAAAAACCAGCCACTCAGCCAGTGAAGAGGAAGTTAGAAGATCTGAGCT
CTGAGTGGAAGGCGGTAAACCGTTTACTTCAAGAGCTGAGGGCAAAGCAG
CCTGACCTAGCTCCTGGACTGACCACTATTGGAGCCTCTCCTACTCAGAC
TGTTACTCTGGTGACACAACCTGTGGTTACTAAGGAAACTGCCATCTCCA
AACTAGAAATGCCATCTTCCTTGATGTTGGAGGTACCTGCTCTGGCAGAT
TTCAACCGGGCTTGGACAGAACTTACCGACTGGCTTTCTCTGCTTGATCA
AGTTATAAAATCACAGAGGGTGATGGTGGGTGACCTTGAGGATATCAACG
AGATGATCATCAAGCAGAAGGCAACAATGCAGGATTTGGAACAGAGGCGT
CCCCAGTTGGAAGAACTCATTACCGCTGCCCAAAATTTGAAAAACAAGAC
CAGCAATCAAGAGGCTAGAACAATCATTACGGATCGAATTGAAAGAATTC
AGAATCAGTGGGATGAAGTACAAGAACACCTTCAGAACCGGAGGCAACAG
TTGAATGAAATGTTAAAGGATTCAACACAATGGCTGGAAGCTAAGGAAGA
AGCTGAGCAGGTCTTAGGACAGGCCAGAGCCAAGCTTGAGTCATGGAAGG
AGGGTCCCTATACAGTAGATGCAATCCAAAAGAAAATCACAGAAACCAAG
CAGTTGGCCAAAGACCTCCGCCAGTGGCAGACAAATGTAGATGTGGCAAA
TGACTTGGCCCTGAAACTTCTCCGGGATTATTCTGCAGATGATACCAGAA
AAGTCCACATGATAACAGAGAATATCAATGCCTCTTGGAGAAGCATTCAT
AAAAGGGTGAGTGAGCGAGAGGCTGCTTTGGAAGAAACTCATAGATTACT
GCAACAGTTCCCCCTGGACCTGGAAAAGTTTCTTGCCTGGCTTACAGAAG
CTGAAACAACTGCCAATGTCCTACAGGATGCTACCCGTAAGGAAAGGCTC
CTAGAAGACTCCAAGGGAGTAAAAGAGCTGATGAAACAATGGCAAGTAAG
TCAGGCATTTCCGCTTTAGCACTCTTGTGGATCCAATTGAACAATTCTCA
GCATTTGTACTTGTAACTGACACCCAGCCTGAAATATGTAGCAAAAT

PCR for gRNA Screen

Screen_F: GAGGCCAAAACAATGCAGAG (SEQ ID NO: 12)

Screen_R: GGAGAGAAAATCTTCTTGACAACAC (SEQ ID NO: 13
)

Primers were chosen such that the forward primer is upstream of all gRNA cut sites in intron 44, and the reverse primer is downstream of all gRNA cut sites in intron 55, such that deletion of the intervening sequence with any guide pair will be amplified with the same primers. Extension times were changed according to the expected deletion amplicon size for each gRNA pair following the manufacturer’s instruction.

PCR Primers for Validation of Deletion and Correct Insertion

HITI B

5′ Insertion Junction

F: TGGAACACAGTTAATTCACTTGG (SEQ ID NO: 14)

R: CCGCAGATTCAGGCTTCC (SEQ ID NO: 15)

3′ Insertion Junction

F: CTGAAACAACTGCCAATGTCC (SEQ ID NO: 16)

R: ACCACCTTAGTTATTCCTCC (SEQ ID NO: 17)

Deletion

F: TGGAACACAGTTAATTCACTTGG (SEQ ID NO: 18)

R: ACCACCTTAGTTATTCCTCC (SEQ ID NO: 19)

HITI K

5′ Insertion Junction

F: GTAGCATAATGGGGTTTCTGC (SEQ ID NO: 20)

R: CCGCAGATTCAGGCTTCC (SEQ ID NO: 21)

3′ Insertion Junction

F: CTGAAACAACTGCCAATGTCC (SEQ ID NO: 22)

R: GCTCAAGTTTTCAGCCACAG (SEQ ID NO: 23)

Deletion

F: GTAGCATAATGGGGTTTCTGC (SEQ ID NO: 24)

R: CCGCAGATTCAGGCTTCC (SEQ ID NO: 25)

HITI B Full sequence

(SEQ ID NO: 26):ctaactagtctagactagctaatgtacaaaaaag
caggctttaaaggaaccaattcagtcgactggatccggtaccaaggtcgg
gcaggaagagggcctatttcccatgattccttcatatttgcatatacgat
acaaggctgttagagagataattagaattaatttgactgtaaacacaaag
atattagtacaaaatacgtgacgtagaaagtaataatttcttgggtagtt
tgcagttttaaaattatgttttaaaatggactatcatatgcttaccgtaa
cttgaaagtatttcgatttcttggctttatatatcttgtggaaaggacga
aacaccgtaggcaagtcagttaaacatgtttaagtactctgtgctggaaa
cagcacagaatctacttaaacaaggcaaaatgccgtgtttatctcgtcaa
cttgttggcgagattttttttataagcttcctgtacagaattcgtctgca
agggcgaattctgcagcgtccctgtacaaaaaagcaggctttaaaggaac
caattcagtcgactggatccggtaccaaggtcgggcaggaagagggccta
tttcccatgattccttcatatttgcatatacgatacaaggctgttagaga
gataattagaattaatttgactgtaaacacaaagatattagtacaaaata
cgtgacgtagaaagtaataatttcttgggtagtttgcagttttaaaatta
tgttttaaaatggactatcatatgcttaccgtaacttgaaagtatttcga
tttcttggctttatatatcttgtggaaaggacgaaacaccgttgtgaagt
tctttccttaggtttaagtactctgtgctggaaacagcacagaatctact
taaacaaggcaaaatgccgtgtttatctcgtcaacttgttggcgagattt
tttttataagcttcctgtacagaattcgtctgcaagggcgaattctgcag
cgtcccactcggatgtttaactgacttgcctacatgtcagtttcataggg
aaattttcacatggagcttttgtatttctttctttgccagtacaactgca
tgtggtagcacactgtttaatcttttctcaaataaaaagacatggggctt
catttttgttttgcctttttggtatcttacaggaactccaggatggcatt
gggcagcggcaaactgttgtcagaacattgaatgcaactggggaagaaat
aattcagcaatcctcaaaaacagatgccagtattctacaggaaaaattgg
gaagcctgaatctgcggtggcaggaggtctgcaaacagctgtcagacaga
aaaaagaggctagaagaacaaaagaatatcttgtcagaatttcaaagaga
tttaaatgaatttgttttatggttggaggaagcagataacattgctagta
tcccacttgaacctggaaaagagcagcaactaaaagaaaagcttgagcaa
gtcaagttactggtggaagagttgcccctgcgccagggaattctcaaaca
attaaatgaaactggaggacccgtgcttgtaagtgctcccataagcccag
aagagcaagataaacttgaaaataagctcaagcagacaaatctccagtgg
ataaaggtttccagagctttacctgagaaacaaggagaaattgaagctca
aataaaagaccttgggcagcttgaaaaaaagcttgaagaccttgaagagc
agttaaatcatctgctgctgtggttatctcctattaggaatcagttggaa
atttataaccaaccaaaccaagaaggaccatttgacgttaaggaaactga
aatagcagttcaagctaaacaaccggatgtggaagagattttgtctaaag
ggcagcatttgtacaaggaaaaaccagccactcagccagtgaagaggaag
ttagaagatctgagctctgagtggaaggcggtaaaccgtttacttcaaga
gctgagggcaaagcagcctgacctagctcctggactgaccactattggag
cctctcctactcagactgttactctggtgacacaacctgtggttactaag
gaaactgccatctccaaactagaaatgccatcttccttgatgttggaggt
acctgctctggcagatttcaaccgggcttggacagaacttaccgactggc
tttctctgcttgatcaagttataaaatcacagagggtgatggtgggtgac
cttgaggatatcaacgagatgatcatcaagcagaaggcaacaatgcagga
tttggaacagaggcgtccccagttggaagaactcattaccgctgcccaaa
atttgaaaaacaagaccagcaatcaagaggctagaacaatcattacggat
cgaattgaaagaattcagaatcagtgggatgaagtacaagaacaccttca
gaaccggaggcaacagttgaatgaaatgttaaaggattcaacacaatggc
tggaagctaaggaagaagctgagcaggtcttaggacaggccagagccaag
cttgagtcatggaaggagggtccctatacagtagatgcaatccaaaagaa
aatcacagaaaccaagcagttggccaaagacctccgccagtggcagacaa
atgtagatgtggcaaatgacttggccctgaaacttctccgggattattct
gcagatgataccagaaaagtccacatgataacagagaatatcaatgcctc
ttggagaagcattcataaaagggtgagtgagcgagaggctgctttggaag
aaactcatagattactgcaacagttccccctggacctggaaaagtttctt
gcctggcttacagaagctgaaacaactgccaatgtcctacaggatgctac
ccgtaaggaaaggctcctagaagactccaagggagtaaaagagctgatga
aacaatggcaagtaagtcaggcatttccgctttagcactcttgtggatcc
aattgaacaattctcagcatttgtacttgtaactgacaagccagggacaa
aacaaaatagttgcttttatacagcctgatgtatttcggtatttggacaa
ggaggagagagctaaggaaagaacttcacaaaagaatataagaatgcggc
cgctaaactat

HITI K Full sequence

(SEQ ID NO: 27)ctaactagtctagactagctaatgtacaaaaaagc
aggctttaaaggaaccaattcagtcgactggatccggtaccaaggtcggg
caggaagagggcctatttcccatgattccttcatatttgcatatacgata
caaggctgttagagagataattagaattaatttgactgtaaacacaaaga
tattagtacaaaatacgtgacgtagaaagtaataatttcttgggtagttt
gcagttttaaaattatgttttaaaatggactatcatatgcttaccgtaac
ttgaaagtatttcgatttcttggctttatatatcttgtggaaaggacgaa
acaccgctgcctgtctcccagtcaaagtttaagtactctgtgctggaaac
agcacagaatctacttaaacaaggcaaaatgccgtgtttatctcgtcaac
ttgttggcgagattttttttataagcttcctgtacagaattcgtctgcaa
gggcgaattctgcagcgtccctgtacaaaaaagcaggctttaaaggaacc
aattcagtcgactggatccggtaccaaggtcgggcaggaagagggcctat
ttcccatgattccttcatatttgcatatacgatacaaggctgttagagag
ataattagaattaatttgactgtaaacacaaagatattagtacaaaatac
gtgacgtagaaagtaataatttcttgggtagtttgcagttttaaaattat
gttttaaaatggactatcatatgcttaccgtaacttgaaagtatttcgat
ttcttggctttatatatcttgtggaaaggacgaaacaccgattttgctac
atatttcagggtttaagtactctgtgctggaaacagcacagaatctactt
aaacaaggcaaaatgccgtgtttatctcgtcaacttgttggcgagatttt
ttttataagcttcctgtacagaattcgtctgcaagggcgaattctgcagc
gtccctttgactgggagacaggcaggggaatagcacactgtttaatcttt
tctcaaataaaaagacatggggcttcatttttgttttgcctttttggtat
cttacaggaactccaggatggcattgggcagcggcaaactgttgtcagaa
cattgaatgcaactggggaagaaataattcagcaatcctcaaaaacagat
gccagtattctacaggaaaaattgggaagcctgaatctgcggtggcagga
ggtctgcaaacagctgtcagacagaaaaaagaggctagaagaacaaaaga
atatcttgtcagaatttcaaagagatttaaatgaatttgttttatggttg
gaggaagcagataacattgctagtatcccacttgaacctggaaaagagca
gcaactaaaagaaaagcttgagcaagtcaagttactggtggaagagttgc
ccctgcgccagggaattctcaaacaattaaatgaaactggaggacccgtg
cttgtaagtgctcccataagcccagaagagcaagataaacttgaaaataa
gctcaagcagacaaatctccagtggataaaggtttccagagctttacctg
agaaacaaggagaaattgaagctcaaataaaagaccttgggcagcttgaa
aaaaagcttgaagaccttgaagagcagttaaatcatctgctgctgtggtt
atctcctattaggaatcagttggaaatttataaccaaccaaaccaagaag
gaccatttgacgttaaggaaactgaaatagcagttcaagctaaacaaccg
gatgtggaagagattttgtctaaagggcagcatttgtacaaggaaaaacc
agccactcagccagtgaagaggaagttagaagatctgagctctgagtgga
aggcggtaaaccgtttacttcaagagctgaggatatcaacgagatgatca
tcaagcagaaggcaacaatgcaggatttggaacagaggcgtccccagttg
gaagaactcattaccgctgcccaaaatttgaaaaacaagaccagcaatca
agaggctagaacaatcattacggatcgaattgaaagaattcagaatcagt
gggatgaagtacaagaacaccttcagaaccggaggcaacagttgaatgaa
atgttaaaggattcaacacaatggctggaagctaaggaagaagctgagca
ggtcttaggacaggccagagccaagcttgagtcatggaaggagggtccct
atacagtagatgcaatccaaaagaaaatcacagaaaccaagcagttggcc
aaagacctccgccagtggcagacaaatgtagatgtggcaaatgacttggc
cctgaaacttctccgggattattctgcagatgataccagaaaagtccaca
tgataacagagaatatcaatgcctcttggagaagcattcataaaagggtg
agtgagcgagaggctgctttggaagaaactcatagattactgcaacagtt
ccccctggacctggaaaagtttcttgcctggcttacagaagctgaaacaa
ctgccaatgtcctacaggatgctacccgtaaggaaaggctcctagaagac
tccaagggagtaaaagagctgatgaaacaatggcaagtaagtcaggcatt
tccgctttagcactcttgtggatccaattgaacaattctcagcatttgta
cttgtaactgacacccagcctgaaatatgtagcaaaatataagaatgcgg
ccgctaaactat

human wild-type DMD exons 45-55

(SEQ ID NO: 28)ACTCGGATGTTTAACTGACTTGCCTACATGTCAGT
TTCATAGGGAAATTTTCACATGGAGCTTTTGTATTTCTTTCTTTGCCAGT
ACAACTGCATGTGGTAGCACACTGTTTAATCTTTTCTCAAATAAAAAGAC
ATGGGGCTTCATTTTTGTTTTGCCTTTTTGGTATCTTACAGGAACTCCAG
GATGGCATTGGGCAGCGGCAAACTGTTGTCAGAACATTGAATGCAACTGG
GGAAGAAATAATTCAGCAATCCTCAAAAACAGATGCCAGTATTCTACAGG
AAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATT
TCAAAGAGATTTAAATGAATTTGTTTTATGGTTGGAGGAAGCAGATAACA
TTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAAT
TCTCAAACAATTAAATGAAACTGGAGGACCCGTGCTTGTAAGTGCTCCCA
TAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAAT
TGAAGCTCAAATAAAAGACCTTGGGCAGCTTGAAAAAAAGCTTGAAGACC
TTGAAGAGCAGTTAAATCATCTGCTGCTGTGGTTATCTCCTATTAGGAAT
CAGTTGGAAATTTATAACCAACCAAACCAAGAAGGACCATTTGACGTTAA
GGAAACTGAAATAGCAGTTCAAGCTAAACAACCGGATGTGGAAGAGATTT
TGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTT
ACTTCAAGAGCTGAGGGCAAAGCAGCCTGACCTAGCTCCTGGACTGACCA
CTATTGGAGCCTCTCCTACTCAGACTGTTACTCTGGTGACACAACCTGTG
GTTACTAAGGAAACTGCCATCTCCAAACTAGAAATGCCATCTTCCTTGAT
GTTGGAGGTACCTGCTCTGGCAGATTTCAACCGGGCTTGGACAGAACTTA
CCGACTGGCTTTCTCTGCTTGATCAAGTTATAAAATCACAGAGGGTGATG
GTGGGTGACCTTGAGGATATCAACGAGATGATCATCAAGCAGAAGGCAAC
AATGCAGGATTTGGAACAGAGGCGTCCCCAGTTGGAAGAACTCATTACCG
CTGCCCAAAATTTGAAAAACAAGACCAGCAATCAAGAGGCTAGAACAATC
ATTACGGATCGAATTGAAAGAATTCAGAATCAGTGGGATGAAGTACAAGA
ACACCTTCAGAACCGGAGGCAACAGTTGAATGAAATGTTAAAGGATTCAA
CACAATGGCTGGAAGCTAAGGAAGAAGCTGAGCAGGTCTTAGGACAGGCC
AGAGCCAAGCTTGAGTCATGGAAGGAGGGTCCCTATACAGTAGATGCAAT
CCAAAAGAAAATCACAGAAACCAAGCAGTTGGCCAAAGACCTCCGCCAGT
GGCAGACAAATGTAGATGTGGCAAATGACTTGGCCCTGAAACTTCTCCGG
GATTATTCTGCAGATGATACCAGAAAAGTCCACATGATAACAGAGAATAT
CAATGCCTCTTGGAGAAGCATTCATAAAAGGGTGAGTGAGCGAGAGGCTG
CTTTGGAAGAAACTCATAGATTACTGCAACAGTTCCCCCTGGACCTGGAA
AAGTTTCTTGCCTGGCTTACAGAAGCTGAAACAACTGCCAATGTCCTACA
GGATGCTACCCGTAAGGAAAGGCTCCTAGAAGACTCCAAGGGAGTAAAAG
AGCTGATGAAACAATGGCAA

OTHER gRNA target sequences

TABLE 1

| | |
|---|---|
| gRNA target pair A | CTAAGGAAAGAACTTCACAA (SEQ ID NO: 6) |
| | TATGCAGCCATAAAAAAAGG (SEQ ID NO: 29) |
| gRNA target pair C | CTAAGGAAAGAACTTCACAA (SEQ ID NO: 6) |
| | CTGCCTGTCTCCCAGTCAAA (SEQ ID NO: 8) |
| gRNA target pair D | CTAAGGAAAGAACTTCACAA (SEQ ID NO: 6) |
| | TTCCTTTTAGGGCAACCTAT (SEQ ID NO: 30) |
| gRNA target pair E | TTAGCTCATATATTGACAGG (SEQ ID NO: 31) |
| | TATGCAGCCATAAAAAAAGG (SEQ ID NO: 29) |
| gRNA target pair F | TTAGCTCATATATTGACAGG (SEQ ID NO: 31) |
| | TAGGCAAGTCAGTTAAACAT (SEQ ID NO: 32) |
| gRNA target pair G | TTAGCTCATATATTGACAGG (SEQ ID NO: 31) |
| | CTGCCTGTCTCCCAGTCAAA (SEQ ID NO: 8) |
| gRNA target pair H | TTAGCTCATATATTGACAGG (SEQ ID NO: 31) |
| | TTCCTTTTAGGGCAACCTAT (SEQ ID NO: 30) |
| gRNA target pair I | ATTTTGCTACATATITCAGG (SEQ ID NO: 9) |
| | TATGCAGCCATAAAAAAAGG (SEQ ID NO: 29) |
| gRNA target pair J | ATTTTGCTACATATTTCAGG (SEQ ID NO: 9) |
| | TAGGCAAGTCAGTTAAACAT (SEQ ID NO: 32) |
| gRNA target pair L | ATTTTGCTACATATTTCAGG (SEQ ID NO: 9) |
| | TTCCTTTTAGGGCAACCTAT (SEQ ID NO: 30) |
| gRNA target pair M | TCTGAAACAGATGCGTTTGG (SEQ ID NO: 33) |
| | TATGCAGCCATAAAAAAAGG (SEQ ID NO: 29) |
| gRNA target pair N | TCTGAAACAGATGCGTTTGG (SEQ ID NO: 33) |
| | TAGGCAAGTCAGTTAAACAT (SEQ ID NO: 32) |
| gRNA target pair O | TCTGAAACAGATGCGTTTGG (SEQ ID NO: 33) |
| | CTGCCTGTCTCCCAGTCAAA (SEQ ID NO: 8) |
| gRNA target pair P | TCTGAAACAGATGCGTTTGG (SEQ ID NO: 33) |
| | TTCCOTTTTAGGGUAACCTAT (SEQ ID NO: 30) |

### Example 2- Development of Innovative Gene Replacement Systems Relevant for DMD Gene Modification in vivo

Applicants are developing precise genomic correction strategies based on CRISPR/Cas9-stimulated homology directed repair (HDR) to enable specific replacement of pathogenic mutations in the Dmd gene with wild-type, non-pathogenic sequences. Applicants have therefore identified three Dmd mutations, mdx, mdx^{4cv}, and mdx^{5cv}, that are targetable within 5 bp of the mutation site by SaCas9, NmeCas9, KKH SaCas9, or SauriCas9.

To screen gRNAs targeting these candidate cut sites for the ability to direct the compatible Cas9 endonuclease activity to the appropriate location, mdx tail-tip fibroblasts (TTFs) were nucleofected with Cas9 orthologs and gRNAs targeting the mdx locus (Table 2). Three days post-nucleofection, genomic DNA was harvested and PCR was performed to amplify the mdx, Pcsk9, and Runx1 loci (Pcsk9 and Runx1 were targeted using previously validated gRNAs and serve as controls, for both cutting activity and specificity, for KKH SaCas9 and SauriCas9, respectively).

TABLE 2

| Condition | Cas9 Ortholog Nucleofected | gRNA Nucleofected | gRNA Protospacer Sequence | |
|---|---|---|---|---|
| 1 | KKH SaCas9 | KKH SaCas9 Runx1 gRNA | GTGTCAGAAGTGTAAGCCCAG | SEQ ID NO: 34 |
| 2 | SauriCas9 | SauriCas9 Pcsk9 gRNA | CCACCGCAGCCACGCAGAGCA | SEQ ID NO: 35 |
| 3 | KKH SaCas9 | KKH SaCas9 mdx gRNA | CAAAGTTCTTTGAAAGAGCAA | SEQ ID NO: 36 |
| 4 | SauriCas9 | SauriCas9 mdx gRNA | GTTCTTTGAAAGAGCAATAAA | SEQ ID NO: 37 |

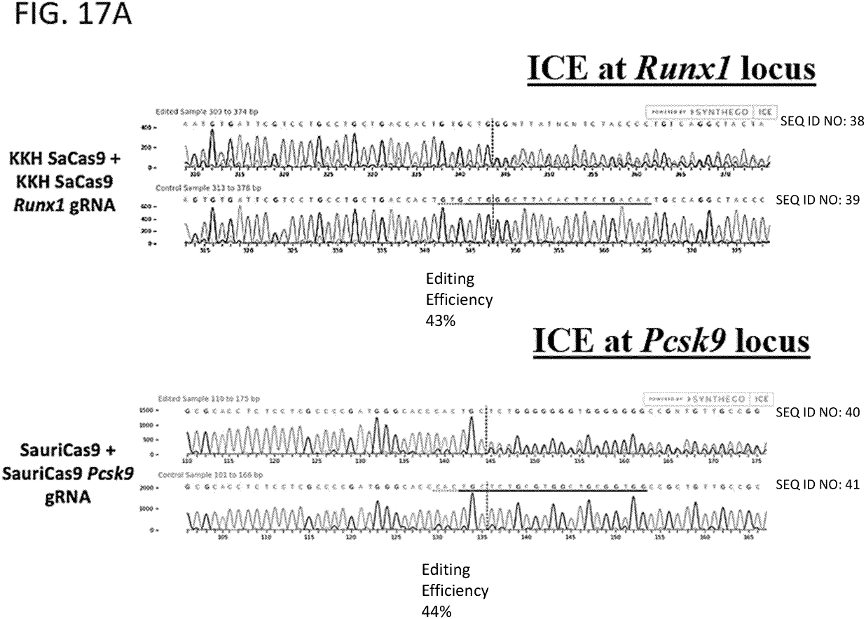

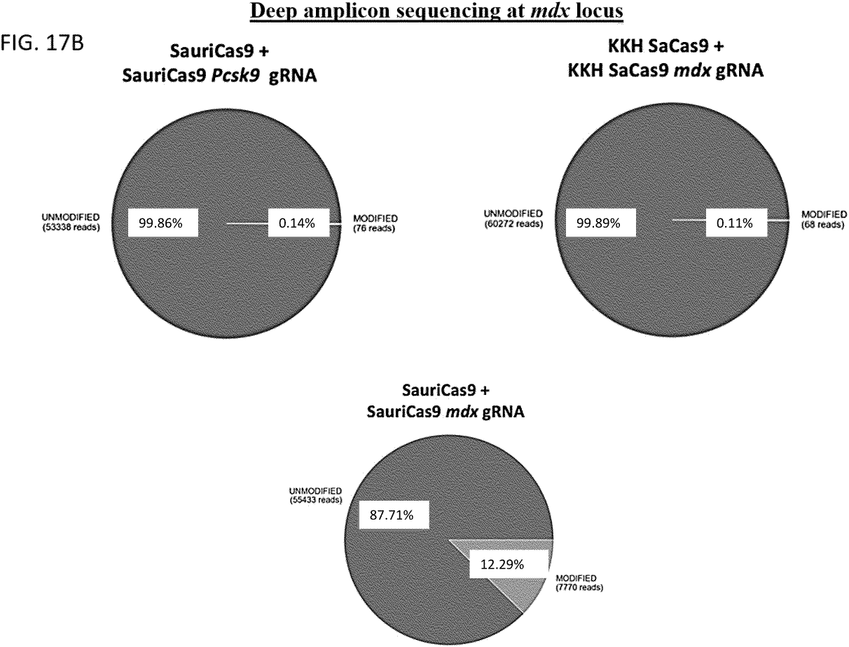

ICE analysis confirmed that both KKH SaCas9 and SauriCas9 were active at the control Pcsk9 and Runx1 loci, respectively (FIG. 17A). Deep amplicon sequencing of exon 23 of the Dmd gene further revealed ~12% of alleles contained indels at the mdx site in the SauriCas9 + SauriCas9 mdx gRNA cells, whereas no indels were detected at the mdx locus when cells were transfected with the control SauriCas9 + SauriCas9 Pcsk9 gRNA cells (FIG. 17B). In contrast, no activity was detected at the mdx locus for KKH SaCas9 + KKH SaCas9 mdx gRNA (FIG. 17B).

Subsequent efforts were focused on the SauriCas9 mdx gRNA. Excitingly, this gRNA directs SauriCas9 cutting 0 bp away from the mdx mutation, making it a very appealing candidate gRNA for optimal HDR efficiency.

### Example 3- Exploring Delivery Mechanisms for Muscle and Satellite Cells

To illuminate the influence that targeting of specific cell types within skeletal muscle has on HDR rates, a system was designed to restrict Cas9 expression to muscle stem cells, also referred to as satellite cells. To accomplish this, the Pax7-CreER^{T2} transgenic mouse strain was cross-bred with the Rosa26-LSL-SpCas9-P2A-EGFP transgenic mouse strain. Once CreER^{T2} is induced by tamoxifen in cells expressing Pax7, SpCas9 and EGFP are expressed bicistronically. Since Pax7 expression is limited to satellite cells within the muscle of postnatal mice, this system restricts SpCas9 and EGFP expression to only satellite cells. Once satellite cells express SpCas9, an SpCas9 gRNA targeting the EGFP locus can be delivered along with a BFP donor template via AAV, allowing a color-switching system to be restricted to satellite cells. This system, when coupled with an analogous system in which SpCas9 and EGFP expression are restricted solely to muscle fibers, enables determination of whether editing in satellite cells is sufficient to yield detectable levels of HDR in muscle fibers.

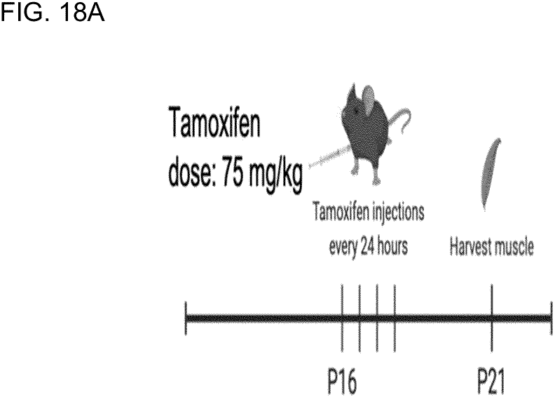

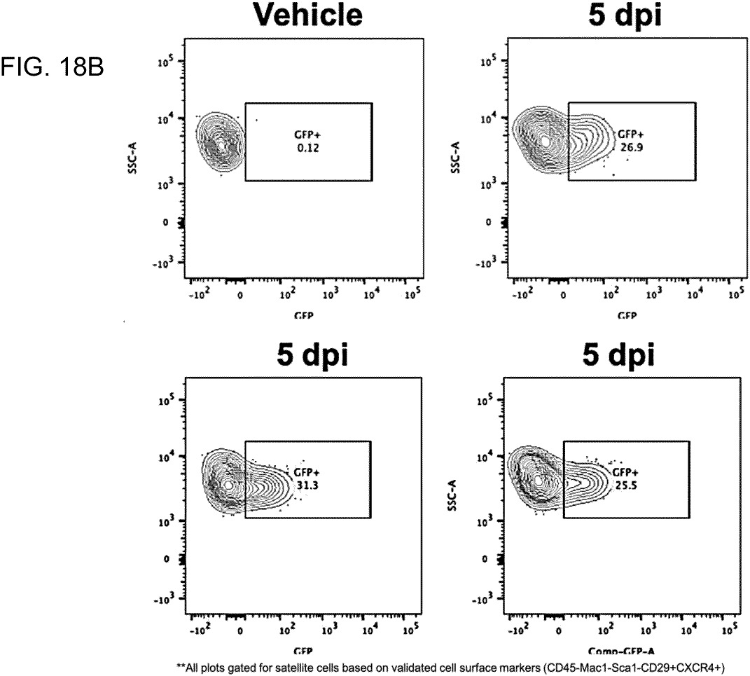

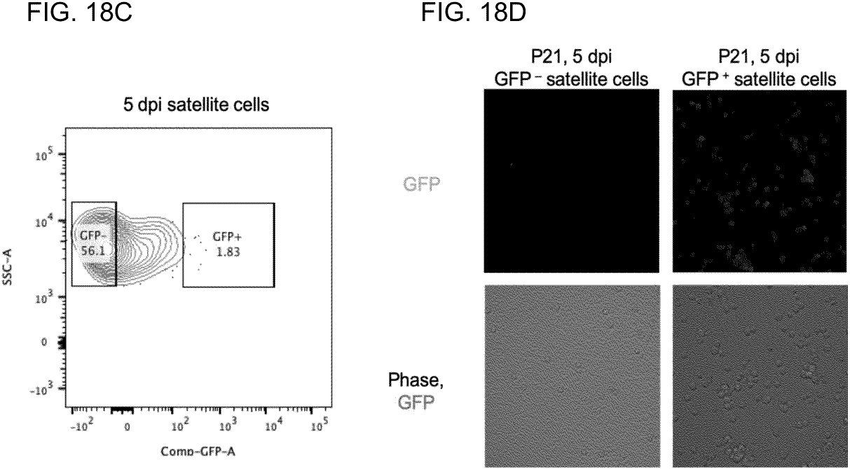

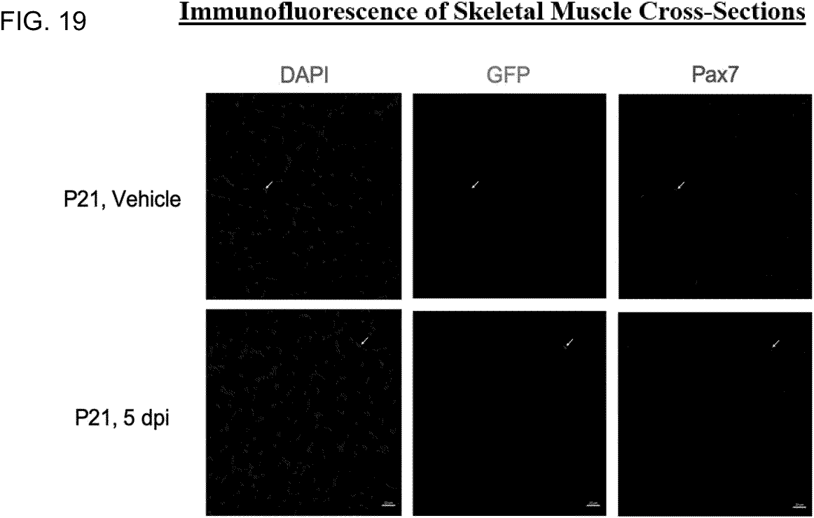

To optimize tamoxifen induction, what dosage and time course of injections would maximize expression of SpCas9 and GFP in satellite cells without resulting in expression in muscle fibers were investigated. mice were injected at P16 for 4 daily injections with 75 mg/kg of tamoxifen with the last one at P19, and then muscle was harvested for satellite cell isolation at P21 (FIG. 18A). FACS analysis indicated that this strategy of daily tamoxifen injections from P16-P19 yielded in detectable GFP fluorescence in 25%-30% of satellite cells analyzed at P21 (FIG. 18B). To confirm these results, two subsets of satellite cells isolated from tamoxifen-injected mice based on their fluorescence profile, either EGFP- or EGFP+ (FIG. 18C) were cultured. Detection of EGFP signal intensity via confocal microscopy confirmed the two subsets of satellite cells were separate populations (FIG. 18D). Knowing that this strategy for tamoxifen injections is sufficient to induce detectable transgene expression in satellite cells, it was next sought to determine whether or not the expression was restricted only to satellite cells or whether EGFP (and therefore also SpCas9) might additionally be detected in myofibers of mice injected at P16-19 and harvested at P21. Applicants therefore cryosectioned, stained for Pax7, and imaged the tibialis anterior (TA) muscles from injected mice. Importantly, no GFP+ myofibers in myofibers of either vehicle- or tamoxifen-injected mice were detected (FIG. 19). Moreover, all GFP+ cells detected in the cryosections colocalized with Pax7 and DAPI counterstains, confirming their satellite cell identity (FIG. 19). These data validate this transgenic system for restricting EGFP and SpCas9 expression to the satellite cell compartment in postnatal skeletal muscle.

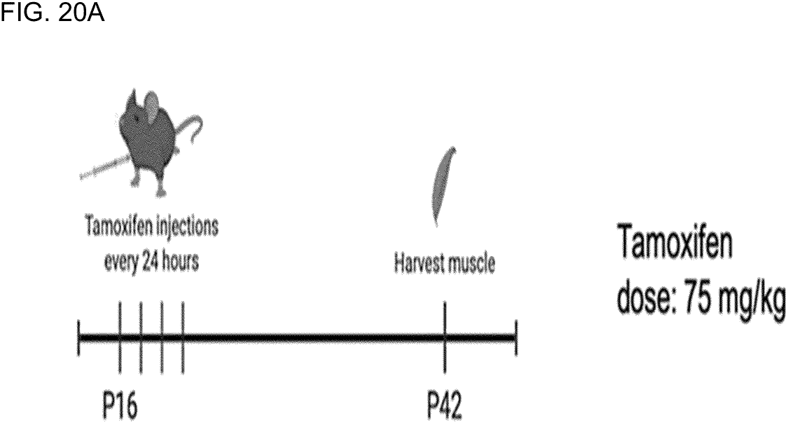

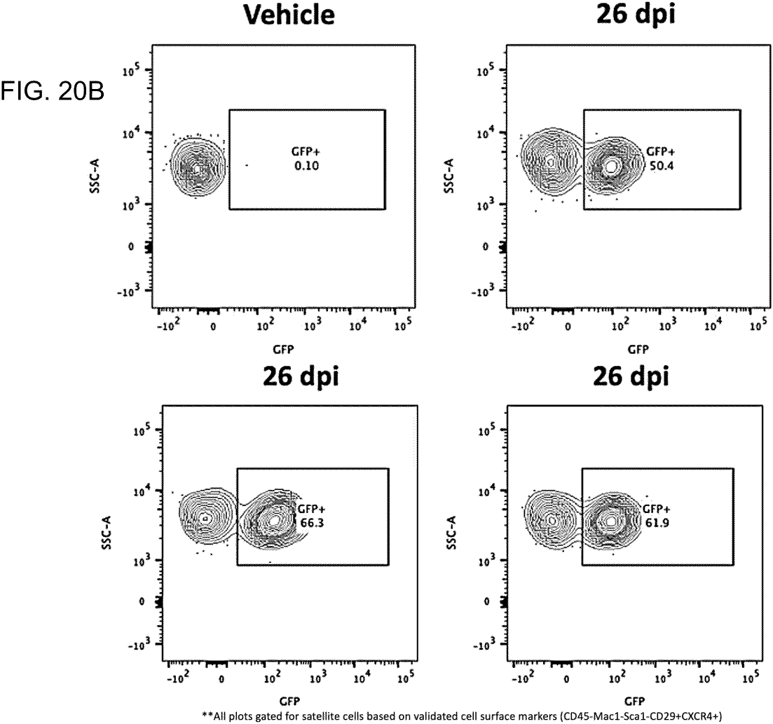

Given that significant tamoxifen induction was observed in the mice after injecting on P16-P19, applicants were interested in determining whether recombination efficiency might increase at later time points post-injection, or if EGFP might be detected within myofibers as well with increasing time after tamoxifen induction (FIG. 20A). A second group of mice (injected at P16-P19) were therefore harvested at P42. FACS analysis demonstrated that daily tamoxifen injection from P16-19 resulted in detectable SpCas9-EGFP in 50%-65% of satellite cells at P42 (FIG. 20B). Histology analysis is being performed.

Given that we have identified a gRNA capable to cutting at the mdx locus, we are proceeding with designing and cloning HDR templates to test the capacity of our CRISPR-AAV-HDR system to precisely correct this mutation in vivo. Although previous studies have determined that longer donor templates typically lead to higher HDR efficiency in vitro, to our knowledge, the effect of varying the length of HDR template has not been explored for in vivo applications. We therefore designed templates with 5 different homology arm lengths and plan to perform intra-muscular injections of these templates along with mdx-targeting SauriCas9/gRNA complexes packaged in AAV. We are currently cloning these templates into AAV-compatible plasmid backbones. In the interim, we have begun to optimize dystrophin staining in preparation for detection of the restoration of dystrophin expression following SauriCas9-directed editing.

To understand whether Cas9 activity is necessary for efficient HDR in muscle fibers, applicants are interested in restricting gene editing to myonuclei while excluding expression in satellite cells. To this end, applicants plan to cross human skeletal actin (HSA)-MerCreMer mice to Rosa26-LSL-SpCas9-P2A-EGFP mice, allowing induction of Cre only in myonuclei while excluding it from muscle progenitor cells. Although these mice are not efficient breeders, applicants have obtained a small cohort of animals and have begun injections of tamoxifen. Applicants plan to perform FACS analysis of satellite cells to confirm that satellite cells do not express Cas9-EGFP and will also collect muscles for cryosectioning and imaging of the myofibers to detect EGFP.

Additionally, applicants are in the process of further optimizing tamoxifen injections in the Pax7-CreER^{T2+/-}; Rosa26-LSL-SpCas9-P2A-EGFP^{+/-} mouse system. By increasing the dose of the tamoxifen (from 75 mg/kg to 100 mg/kg) and the number of injections (from 4 to 5), applicants plan to further increase the efficiency of recombination in order to maximize the opportunity for gene editing events in satellite cells of these mice. To prepare for eventual delivery of an EGFP-targeting gRNA and BFP donor template following tamoxifen induction, applicants are in the process of packaging and producing high-titer batches of AAV encoding each of these components of our HDR system.

What is claimed is:

1. A method for modifying the genome of a mammalian muscle or muscle precursor cell, comprising contacting the cell with a Cas protein and a first and second guide ribonucleic acid (gRNA), wherein the first gRNA hybridizes to a first target site located in intron 44 of DMD and the second gRNA hybridizes to a second target site located in intron 55 of DMD, thereby modifying the genome of the mammalian muscle or muscle precursor cell located between intron 44 and intron 55 of DMD, wherein the genome of the mammalian muscle or muscle precursor cell located between intron 44 and intron 55 of DMD comprises a mutation associated with a disease or condition.

2. The method of claim 1, wherein the modification of the genome comprises a deletion of the nucleotide sequence between intron 44 and intron 55 of DMD.

3. The method of claim 1, further comprising contacting the cell with template DNA comprising the nucleotide sequences of exons 45 to 55 of wild-type DMD.

4. The method of claim 3, wherein the template DNA comprises portions of the first and second target sites flanking the nucleotide sequences of exons 45 to 55 of wild-type DMD.

5. The method of claim 3, wherein the modification of the genome comprises replacement of the nucleotide sequence between intron 44 and intron 55 of DMD with template DNA comprising exons 45 to 55 of wild-type DMD.

6. The method of claim 5, wherein the replacement of the nucleotide sequence between intron 44 and intron 55 of DMD occurs via non-homologous end joining (NHEJ).

7. (canceled)

8. The method of claim 1, wherein the disease or condition is Duchenne muscular dystrophy.

9. The method of claim 1, wherein the cell is a human cell.

10. The method of claim 1, wherein the cell is an induced pluripotent stem cell derived from a cell of a subject with a muscular dystrophy and having a mutation located between intron 44 and intron 55 of the DMD gene.

11-12. (canceled)

13. The method of claim 1, wherein the cell is contacted with one or more viruses transducing the Cas protein, the first gRNA, the second gRNA, or the template DNA.

14. The method of claim 13, wherein the one or more viruses are AAV viruses.

15. The method of claim 1, wherein the cell is contacted with a first virus transducing a nucleic acid encoding the Cas protein in the cell and a second virus transducing a nucleic acid encoding the first gRNA, the second gRNA, and template DNA in the cell.

16. The method of claim 15, wherein at least the first virus or second virus is an AAV virus.

17. The method of claim 1, wherein the cell with a modified genome expresses truncated functional dystrophin lacking an amino acid sequence coded by exons 45 to 55 of wild-type DMD.

18. The method of claim 3, wherein the cell with a modified genome expresses full length dystrophin comprising amino acid sequences coded by exons 45 to 55 of wild-type DMD.

19. The method of claim 1 wherein the cell is contacted in vitro, ex vivo, or in vivo.

20. The method of claim 1, wherein the cell is contacted with a virus inducing a nucleic acid of SEQ ID NO: 26 or 27, or a portion thereof inducing one or more of one or two gRNAs and a template.

21. A method of treating a muscular dystrophy in a subject in need thereof, comprising contacting a muscle or muscle precursor cell of the patient with a Cas protein and a first and second guide ribonucleic acid (gRNA), wherein the first gRNA hybridizes to a first target site located in intron 44 of DMD and the second gRNA hybridizes to a second target site located in intron 55 of DMD, thereby modifying the genome of the cell, wherein the subject’s genome comprises a mutation located between intron 44 and intron 55 of the DMD gene.

22-36. (canceled)

37. A method for modifying the genome of a mammalian muscle or muscle precursor cell, comprising contacting the cell with a Cas protein and a first and second guide ribonucleic acid (gRNA), wherein the first gRNA hybridizes to a first target site located in a first intron of DMD and the second gRNA hybridizes to a second target site located in another intron of DMD, thereby modifying the genome of the mammalian muscle or muscle precursor cell located between the first and second target sites.

38-51. (canceled)

52. A composition comprising a first virus transducing a nucleic acid encoding a Cas protein in a cell and a second virus transducing a nucleic acid encoding a first gRNA, a second gRNA, and a template DNA in a cell, wherein the first gRNA hybridizes to a first target site located in a first intron of DMD and the second gRNA hybridizes to a second target site located in another intron of DMD, and wherein the template DNA codes for one or more DMD exons located between the first and second targets sites.

53. (canceled)


SEQUENCE LISTING (41 sequences)

SEQ ID NO: 1 - DNA, 46 bp, Artificial
  synthetic construct
gtttcctttaattcttttgttttaactgactttgcctaaaagccac

SEQ ID NO: 2 - DNA, 44 bp, Artificial
  synthetic construct
atgaattttgctacatatttcgactgggagacaggcaggggaat

SEQ ID NO: 3 - DNA, 87 bp, Homo sapiens
gtatttggacaaggaggagagagcacaagttgaaattaaactacaccaaggtgaacttaatttgtgttgtcaagaagattttctctc

SEQ ID NO: 4 - DNA, 73 bp, Homo sapiens
aatagggaaacagcatatggatgataaccagtgtattatagcatcatacatatagcatgtcagtttcataggg

SEQ ID NO: 5 - DNA, 70 bp, Artificial
  synthetic construct
agggaaacagcatatggatgataaccagtgccaaggtgaacttaatttgtgttgtcaagaagattttctc

SEQ ID NO: 6 - DNA, 20 bp, Artificial
  gRNA target
ctaaggaaagaacttcacaa

SEQ ID NO: 7 - DNA, 20 bp, Artificial
  gRNA target
ttgtgaagttctttccttag

SEQ ID NO: 8 - DNA, 20 bp, Artificial
  gRNA target
ctgcctgtctcccagtcaaa

SEQ ID NO: 9 - DNA, 20 bp, Artificial
  gRNA target
attttgctacatatttcagg

SEQ ID NO: 10 - DNA, 2131 bp, Artificial
  DNA template
actcggatgtttaactgacttgcctacatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtcaggcatttccgctttagcactcttgtggatccaattgaacaattctcagcatttgtacttgtaactgacaagccagggacaaaacaaaatagttgcttttatacagcctgatgtatttcggtatttggacaaggaggagagagctaaggaaagaacttcacaaaagaat

SEQ ID NO: 11 - DNA, 1881 bp, Artificial
  DNA template
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtcaggcatttccgctttagcactcttgtggatccaattgaacaattctcagcatttgtacttgtaactgacacccagcctgaaatatgtagcaaaat

SEQ ID NO: 12 - DNA, 20 bp, Artificial
  primer
gaggccaaaacaatgcagag

SEQ ID NO: 13 - DNA, 25 bp, Artificial
  primer
ggagagaaaatcttcttgacaacac

SEQ ID NO: 14 - DNA, 22 bp, Artificial
  primer
tggaacacagttaattcacttg

SEQ ID NO: 15 - DNA, 18 bp, Artificial
  primer
ccgcagattcaggcttcc

SEQ ID NO: 16 - DNA, 21 bp, Artificial
  primer
ctgaaacaactgccaatgtcc

SEQ ID NO: 17 - DNA, 20 bp, Artificial
  primer
accaccttagttattcctcc

SEQ ID NO: 18 - DNA, 23 bp, Artificial
  primer
tggaacacagttaattcacttgg

SEQ ID NO: 19 - DNA, 20 bp, Artificial
  primer
accaccttagttattcctcc

SEQ ID NO: 20 - DNA, 21 bp, Artificial
  primer
gtagcataatggggtttctgc

SEQ ID NO: 21 - DNA, 18 bp, Artificial
  primer
ccgcagattcaggcttcc

SEQ ID NO: 22 - DNA, 21 bp, Artificial
  primer
ctgaaacaactgccaatgtcc

SEQ ID NO: 23 - DNA, 20 bp, Artificial
  primer
gctcaagttttcagccacag

SEQ ID NO: 24 - DNA, 21 bp, Artificial
  primer
gtagcataatggggtttctgc

SEQ ID NO: 25 - DNA, 18 bp, Artificial
  primer
ccgcagattcaggcttcc

SEQ ID NO: 26 - DNA, 3145 bp, Artificial
  synthetic construct
ctaactagtctagactagctaatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaggcaagtcagttaaacatgtttaagtactctgtgctggaaacagcacagaatctacttaaacaaggcaaaatgccgtgtttatctcgtcaacttgttggcgagattttttttataagcttcctgtacagaattcgtctgcaagggcgaattctgcagcgtccctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgtgaagttctttccttaggtttaagtactctgtgctggaaacagcacagaatctacttaaacaaggcaaaatgccgtgtttatctcgtcaacttgttggcgagattttttttataagcttcctgtacagaattcgtctgcaagggcgaattctgcagcgtcccactcggatgtttaactgacttgcctacatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtcaggcatttccgctttagcactcttgtggatccaattgaacaattctcagcatttgtacttgtaactgacaagccagggacaaaacaaaatagttgcttttatacagcctgatgtatttcggtatttggacaaggaggagagagctaaggaaagaacttcacaaaagaatataagaatgcggccgctaaactat

SEQ ID NO: 27 - DNA, 2997 bp, Artificial
  synthetic construct
ctaactagtctagactagctaatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgcctgtctcccagtcaaagtttaagtactctgtgctggaaacagcacagaatctacttaaacaaggcaaaatgccgtgtttatctcgtcaacttgttggcgagattttttttataagcttcctgtacagaattcgtctgcaagggcgaattctgcagcgtccctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgattttgctacatatttcagggtttaagtactctgtgctggaaacagcacagaatctacttaaacaaggcaaaatgccgtgtttatctcgtcaacttgttggcgagattttttttataagcttcctgtacagaattcgtctgcaagggcgaattctgcagcgtccctttgactgggagacaggcaggggaatagcacactgtttaatcttttctcaaataaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagtaagtcaggcatttccgctttagcactcttgtggatccaattgaacaattctcagcatttgtacttgtaactgacacccagcctgaaatatgtagcaaaatataagaatgcggccgctaaactat

SEQ ID NO: 28 - DNA, 1955 bp, Homo sapiens
actcggatgtttaactgacttgcctacatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaa

SEQ ID NO: 29 - DNA, 20 bp, Artificial
  gRNA target
tatgcagccataaaaaaagg

SEQ ID NO: 30 - DNA, 20 bp, Artificial
  gRNA target
ttccttttagggcaacctat

SEQ ID NO: 31 - DNA, 20 bp, Artificial
  gRNA target
ttagctcatatattgacagg

SEQ ID NO: 32 - DNA, 20 bp, Artificial
  gRNA target
taggcaagtcagttaaacat

SEQ ID NO: 33 - DNA, 20 bp, Artificial
  gRNA target
tctgaaacagatgcgtttgg

SEQ ID NO: 34 - DNA, 21 bp, Artificial
  gRNA target
gtgtcagaagtgtaagcccag

SEQ ID NO: 35 - DNA, 21 bp, Artificial
  gRNA target
ccaccgcagccacgcagagca

SEQ ID NO: 36 - DNA, 20 bp, Artificial
  gRNA target
caagttctttgaaagagcaa

SEQ ID NO: 37 - DNA, 21 bp, Artificial
  gRNA target
gttctttgaaagagcaataaa

SEQ ID NO: 38 - DNA, 65 bp, Artificial
  synthetic construct
  misc_feature (38)..(38): n is a, c, g, or t
  misc_feature (42)..(42): n is a, c, g, or t
  misc_feature (44)..(44): n is a, c, g, or t
aatgtgattcgtcctgcctgctgaccactgtgctgggnttancntctacccctgtcaggctacta

SEQ ID NO: 39 - DNA, 66 bp, Artificial
  synthetic construct
agtgtgattcgtcctgcctgctgaccactgtgctgggcttacacttctgacactgccaggctaccc

SEQ ID NO: 40 - DNA, 66 bp, Artificial
  synthetic construct
  misc_feature (57)..(57): n is a, c, g, or t
gcgcacctctcctcgccccgatgggcacccactgctctgggggggtgggggggccgntgttgccgg

SEQ ID NO: 41 - DNA, 66 bp, Artificial
  synthetic construct
gcgcacctctcctcgccccgatgggcacccactgctctgcgtggctgcggtggccgctgttgccgc